(19) United States
(12) Patent Application Publication
(10) Pub. No.: US 2020/0332252 A1
(43) Pub. Date: Oct. 22, 2020

(54) HOLLOW MICROCARRIER FOR SHEAR-FREE CULTURE OF ADHERENT CELLS IN BIOREACTORS

(71) Applicant: Board of Supervisors of Louisiana State University and Agricultural and Mechanical College, Baton Rouge, LA (US)

(72) Inventors: Kidong Park, Baton Rouge, LA (US); Edward Song, Baton Rouge, LA (US)

(21) Appl. No.: 16/755,650

(22) PCT Filed: Oct. 11, 2018
(86) PCT No.: PCT/US2018/055352

§ 371 Date: Apr. 13, 2020

Related U.S. Application Data
(60) Provisional application No. 62/571,336, filed on Oct. 12, 2017.

(51) Int. Cl. C12N 5/00

(52) U.S. Cl. CPC C12N 5/0075; C12N 2533/00; C12N 2531/00

(57) ABSTRACT

The present invention provides hollow microcarriers for cell culture. The hollow microcarriers form a shell around a hollow interior and can be opened to permit cell infiltration or harvesting. The hollow microcarriers protect cells from hydrodynamic shear stress without hindering the diffusion of nutrients in and out of their hollow interior.

### CROSS-REFERENCE TO RELATED APPLICATIONS

This application claims priority to U.S. Provisional Patent Application No. 62/571,336, filed Oct. 12, 2017, which is incorporated by reference herein in its entirety.

### BACKGROUND OF THE INVENTION

With recent advances in biotechnologies and cell-based therapies, there are strong needs for efficient and reliable platforms to expand adherent cells in a large quantity. In biopharmaceutical industries, complex protein therapeutics and monoclonal antibodies are produced by various living organisms (Butler M, Applied microbiology and biotechnology, 2005, 68(3):283-291; Warnock J N et al., Biotechnology and applied biochemistry, 2006, 45(1):1-12; Wurm F M, Nature biotechnology, 2004, 22(11):1393). Mammalian cells are often the preferred expression systems for producing complex biopharmaceutics because they possess more human-compatible post-transcriptional metabolic machinery (Wurm F M, Nature biotechnology, 2004, 22(11):1393; Sethuraman N et al., Current opinion in biotechnology, 2006, 17(4):341-346). Furthermore, a large amount of functional stem cells are required for various cell-based therapies, which show a great potential to provide permanent cures for degenerative diseases (Daley G Q et al., Cell, 2008, 132(4):544-548; Segers V F M et al., Nature, 2008, 451(7181):937). Recent studies report efficacy of cell-based therapies on cardiac disease (Segers V F M et al., Nature, 2008, 451(7181):937), cartilage repair (Brittberg M, The American journal of sports medicine, 2010, 38(6):1259-1271), neurological disorder (Kim S U et al., Journal of neuroscience research, 2009, 87(10):2183-2200), bone damage and diseases (Cancedda R et al., Stem Cells, 2003, 21(5):610-619), arthritis (Augello A et al., Arthritis & Rheumatology, 2007, 56(4):1175-1186), and others (Daley G Q et al., Cell, 2008, 132(4):544-548). These stem cells are sensitive to hydrodynamic shear stress and are more challenging to expand on a large scale (Wurm FM, Nature biotechnology, 2004, 22(11):1393; Sethuraman N et al., Current opinion in biotechnology, 2006, 17(4):341-346; Dunlop E H et al., Chemical Engineering Science, 1994, 49(14):2263-2276; Xing Z et al., Biotechnology and bioengineering, 2009, 103(4):733-746). In laboratories, adherent cells are typically cultured using culture flasks having culture areas of about 25-175 cm². However, large-scale cell expansion often requires over hundreds or thousands of such culture flasks, which are impractical due to the amount of required labor. Roller bottles (Liu Y L et al., Biotechniques, 2003, 34(1):184-189) or multilayer planar vessels (U.S. Pat. No. 8,178,345, the contents of which are incorporated herein in its entirety) can be used to provide much larger growth areas of about 1,000-10,000 cm². Using these to expand adherent cells tends to be an easy and direct translation from culture flasks, but they are still limited in their scale-up potential.

Currently, for a large scale culture of adherent cells, a number of different platforms are available, such as microcarrier-based stirred bioreactors (Eibes G et al., Journal of biotechnology, 2010, 146(4):194-197; Hu A Y C et al., Vaccine, 2008, 26(45):5736-5740; Lundgren B et al., Bioseparation and Bioprocessing: Biochromatography, Membrane Separations, Modeling, Validation, 1998, 165-222; Nam J H et al., Biotechnology progress, 2007, 23(3):652-660), packed-bed bioreactors (Looby D et al., Cytotechnology, 1988, 1(4):339-346), fluidized-bed bioreactors (Keller J et al., Advances in Bioprocess Engineering. Springer Netherlands, 1994, 115-121), and hollow fiber bioreactors (Ku K et al., Biotechnology and Bioengineering, 1981, 23(1):79-95). Among these, the microcarrier-based stirred bioreactors are widely used to culture cells that cannot survive as single cells or cell aggregates. Such bioreactors grow the anchorage dependent cells on the outer surfaces of suspended microcarriers, which are essentially solid microspheres. The microcarrier-based stirred bioreactor can support large capacities and massive quantities of anchorage dependent cells can be produced in a single run.

As the capacity of a bioreactor increases, more vigorous stirring and aeration are necessary to maintain sufficient mass transfer rates of nutrients and gases for the larger number of cells (Xing Z et al., Biotechnology and bioengineering, 2009, 103(4):733-746). However, this increases hydrodynamic shear stress that can lead to adverse effects on cells, such as reduced proliferation, low viability, and uncontrolled differentiation of stem cells (Croughan M S et al., Biotechnology and bioengineering, 1987, 29(1):130-141; Gupta Pet al., Cytotechnology, 2016, 68(1):45-59; Leung H W et al., Tissue Engineering Part C: Methods, 2010, 17(2):165-172; Ng Y C et al., Biotechnology and bioengineering, 1996, 50(6):627-635; O'Connor K C et al., Biotechnology techniques, 1992, 6(4):323-328). The trade-off between the mass transfer rate and the hydrodynamic shear stress makes large-scale expansion of shear-sensitive cells unreliable and leads to time-consuming optimization of operating conditions on each expansion stages, as those factors are typically affected by the bioreactor's capacity.

One of the approaches to address this issue is to optimize configuration and geometry of stirred bioreactors and their impellers for maximum media mixing and minimum hydrodynamic shear stress. Numerous studies were able to make improvements to a certain degree, yet they could not overcome the fundamental limits imposed by the finite diffusion rate of gases and nutrients and the hydrodynamics (Dusting J et al., Biotechnology and bioengineering, 2006, 94(6):1196-1208; Odeleye A O O et al., Chemical engineering science, 2014, 111:299-312; Cioffi M et al., Journal of biomechanics, 2008, 41(14):2918-2925; Sucosky P et al., Biotechnology and bioengineering, 2004, 85(1):34-46; Santiago P A et al., Process biochemistry, 2011, 46(1):35-45; Grein T A et al., Process Biochemistry, 2016, 51(9):1109-1119). Another approach is to locally shield cells from the hydrodynamic shear stress. This approach includes the use of macroporous microcarrier (Ng Y C et al., Biotechnology and bioengineering, 1996, 50(6):627-635; Nilsson K et al., Nature Biotechnology, 1986, 4(11):989-990), fiber discs in packed-bed reactors (Meuwly F et al., Biotechnology and bioengineering, 2006, 93(4):791-800; Petti S A et al., Biotechnology progress, 1994, 10(5):548-550), and various encapsulation methods (Bauwens C et al., Biotechnology and Bioengineering, 2005, 90(4):452-461; Jing D et al., Cell transplantation, 2010, 19(11):1397-1412). Generally, in these techniques, cells are placed inside of microstructures to be protected from the hydrodynamic shear stress (Martens D E et al., Cytotechnology, 1996, 21(1):45-59). However, such protection from the external flow make it difficult for nutrients and gases to be uniformly available to the cells, as some of them are located deep inside the protective structures (Preissmann A et al., Cytotechnology, 1997, 24(2):121-134). Also, for the very same reason, the cell harvesting is very challenging.

Therefore, there is a need for improved microcarriers that are capable of culturing adherent cells in bioreactors while shielding the cells from hydrodynamic shear stress. The present invention addresses this need.

### SUMMARY OF THE INVENTION

In one aspect, the present invention relates to a hollow microcarrier comprising a thin shell forming a three-dimensional structure having a hollow interior, the structure having a shape selected from the group consisting of: a sphere, an elongated sphere, a cylinder, a spheroid, and a polyhedron. In one embodiment, the shell comprises one or more holes, gaps, or apertures accessing the hollow interior.

In one embodiment, the shell comprises a plurality of elongate leaflets, each leaflet having a proximal end and a distal end, wherein the plurality of leaflets are joined to each other at their proximal ends in a radial pattern, and wherein the distal ends of the plurality of leaflets curl towards each other to form a substantially spherical shape having a hollow interior. In one embodiment, the hollow microcarrier comprises between 3 and 10 leaflets.

In one embodiment, the shell comprises a plurality of elongate leaflets, each leaflet having a proximal end and a distal end, wherein the plurality of leaflets are joined to each other at their proximal ends in a first and a second radial pattern, wherein the first and second radial patterns are joined to each other by the distal end of a leaflet, and wherein the distal ends of the leaflets curl towards each other such that the first radial pattern and the second radial pattern each form a hemisphere of a substantially spherical shape having a hollow interior.

In one embodiment, the shell comprises a plurality of elongate leaflets, each leaflet defining a gore segment having opposing ends and a central region, wherein each leaflet is joined to an adjacent leaflet at the central region in a linear array, and wherein the opposing ends of the leaflets curve towards each other to form a substantially spherical shape having a hollow interior.

In one embodiment, the shell comprises a rectangular leaflet joined to two circular leaflets, wherein the leaflets curve towards each other such that the rectangular leaflet forms a curved outer surface and the circular leaflets form opposing ends of a substantially cylindrical shape having a hollow interior.

In one embodiment, the shell comprises a plurality of polygonal leaflets joined to each other, wherein the leaflets curve towards each other to form a substantially polyhedral shape having a hollow interior.

In one embodiment, the hollow microcarrier is constructed from a layer of a first material bonded to a layer of a second material. In one embodiment, the layer of the first material and the layer of the second material are under different amounts of tensile stress or different amounts of compressive stress. In one embodiment, the different amounts of tensile stress or different amounts of compressive stress are caused by the layer of the first material and the layer of the second material having different coefficients of thermal expansion. In one embodiment, the different amounts of tensile stress or different amounts of compressive stress are caused by the layer of the first material and the layer of the second material being fabricated at different processing temperatures. In one embodiment, the different amounts of tensile stress or different amounts of compressive stress are caused by the layer of the first material and the layer of the second material having different swelling ratios. In one embodiment, the hollow microcarrier has a diameter between about 50 μm and 10 mm.

In one embodiment, the first material is a mix of Sylgard 184 and Sylgard 3-6636 in a 5:1:3:3 ratio of Base _{Sylgard 184}: Curing agent _{Sylgard 184}: Part-A _{Sylgard 3-6636}: Part-B _{Sylgard 3-6636}. In one embodiment, the second material is a mix of Sylgard 184 and Xiameter-200M in a 4:1:1 ratio of Base _{Sylgard 184}. Curing agent _{Sylgard 184}: Xiameter.

In one embodiment, the shell comprises one or more markings selected from the group consisting of: letters, numbers, shapes, symbols, barcodes, Quick Response (QR) codes, images, and combinations thereof.

In another aspect, the present invention relates to a method of fabricating hollow microcarriers, the method comprising the steps of: depositing a layer of sacrificial material on a substrate; depositing a layer of a first material on the layer of sacrificial material; depositing a layer of a second material on the layer of the first material; engraving one or more hollow microcarrier patterns into the layer of sacrificial material, the layer of the first material, and the layer of the second material; applying one or more surface treatments to the layer of the second material; and removing the layer of sacrificial material to release the layer of the first material and the layer of the second material from the substrate.

In one embodiment, the sacrificial material is AZ-9260 photoresist spin-coated on a substrate at 1300 rpm for 10 seconds to achieve a layer thickness of 13 μm and baked at 140° C. for 1 hour. In one embodiment, the substrate is a flat piece of silicon.

In one embodiment, the first material is a mix of Sylgard 184 and Sylgard 3-6636 in a 5:1:3:3 ratio of Base _{Sylgard 184}: Curing agent _{Sylgard 184}: Part-A _{Sylgard 3-6636}: Part-B _{Sylgard 3-6636} that is spin-coated on the sacrificial material at 2000 rpm for 3 minutes to achieve a layer thickness of 18μm and baked at 40° C. for 12 hours.

In one embodiment, the second material is a mix of Sylgard 184 and Xiameter-200M in a 4:1:1 ratio of Base _{Sylgard 184}: Curing agent _{Sylgard 184}: Xiameter that is spin-coated on the first material at 1300 rpm for 2 minutes to achieve a layer thickness of 19 μm and baked at 130° C. for 3 hours.

In one embodiment, the one or more surface treatments includes a corona discharge treatment that renders portions of the hollow microcarrier hydrophilic or hydrophobic. In one embodiment, the one or more surface treatments includes a coating of a cell growth promoting composition.

In one embodiment, the method further comprises a step of applying one or more markings on the sacrificial material, the first material, the second material, and combinations thereof using photolithography, stereolithography, or laser etching. In one embodiment, the one or more markings are selected from the group consisting of: letters, numbers, shapes, symbols, barcodes, Quick Response (QR) codes, images, and combinations thereof.

In another aspect, the present invention relates to a method of culturing cells using hollow microcarriers, the method comprising the steps of: adding an amount of hollow microcarriers to a suspension of cells; shifting the hollow microcarriers between a closed configuration, an open configuration, and back to a closed configuration in the suspension of cells to introduce cells into the hollow microcarriers; incubating the hollow microcarriers under static conditions; incubating the hollow microcarriers under dynamic conditions; and shifting the hollow microcarriers from a closed configuration to an open configuration to harvest the cells from the hollow microcarriers.

In one embodiment, the hollow microcarriers are shifted between the open configuration and the closed configuration using thermal actuation or mechanical force.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A through FIG. 1D depict exemplary hollow microcarriers (HMCs) of the present invention.

FIG. 2 is a flowchart depicting an exemplary method of fabricating the HMCs of the present invention.

FIG. 3 is a flowchart depicting an exemplary method of culturing cells using the HMCs of the present invention.

FIG. 4 illustrates the working principle of HMCs. (a) The cells on conventional microcarriers are exposed to excessive shear stress. (b) The HMCs culture cells inside a hollow enclosure, protecting the cells from shear stress.

FIG. 5A through FIG. 5D depict the fabrication process and seeding procedure of exemplary HMCs. (FIG. 5A) A schematic diagram of the HMC fabrication process and surface treatment. (FIG. 5B) Exemplary surface functionalization scheme of HMCs for NIH/3T3 fibroblasts. (FIG. 5C) Exemplary surface functionalization scheme of HMCs for hiPSCs. (FIG. 5D) HMCs are seeded as they pass through a narrow orifice with cells. (scale bar: 2 cm).

FIG. 6A through FIG. 6H depict the geometry of exemplary HMCs: (FIG. 6A) double hemisphere pattern; (FIG. 6B) linear pattern; (FIG. 6C) snowflake pattern; (FIG. 6D) snowflake pattern modified by shortening the ends of the leaflets; (FIG. 6E) snowflake pattern modified with side holes; (FIG. 6F) snowflake pattern modified by increasing the gap between the leaflets. (FIG. 6G) The size of the HMCs can be controlled with PDMS film thickness. (FIG. 6H) HMCs with radii of 426 μm, 573 μm, and 864 μm. (scale bars: 300 μm).

FIG. 7A through FIG. 7C depict the results of numerical analysis on shear stress and glucose diffusion in the HMCs. (FIG. 7A) Shear stress plot; the left half of the HMC shows the shear stress on the external surface, the right half shows the shear stress on the internal surface. (FIG. 7B) Concentration of glucose inside an HMC is decreased by 2%. (FIG. 7C) The reduction of the average shear stress and the drop in the glucose concentration can be fine-tuned with the opening angle of the HMC.

FIG. 8A through FIG. 8C depict the results of HMC surface treatment. (FIG. 8A) Phase-contrast image of HMCs treated with 2-[Methoxy(Polyethyleneoxy)6-9Propyl]Trimethoxysilane (MPEGTMS) (left) and without treatment (right). (scale bar: 200 μm). (FIG. 8B) SEM image of an HMC with MPEGTMS treatment. (FIG. 8C) SEM image of an HMC without treatment. Note that the HMC with MPEGTMS treatment only has cells adhered to its interior, while the HMC without treatment has cells adhered to its interior and exterior. (scale bar: 500 μm)

FIG. 9A through FIG. 9D depict the results of fibroblast growth and morphology in the HMCs. (FIG. 9A) Fibroblast 3T3 shows active growth with the HMCs over 6 days. (scale bar: 300 μm). (FIG. 9B) Cell morphology is shown on each day. (scale bar: 100 μm). (FIG. 9C) Fibroblast 3T3 were expanded in HMCs with different stirring rates and cell numbers were counted using CCK-8 on each day. (FIG. 9D) Fold increases of Fibroblast 3T3 with HMCs with different stirring rates are presented.

FIG. 10A through FIG. 10F depict the results of hiPSC growth and differentiation with HMCs. (FIG. 10A) Bright-field images of hiPSC seeded HMCs under dynamic (30 rpm) or static culture conditions. (scale bar: 200 μm; inset scale bar: 50 μm). (FIG. 10B) hiPSC growth in HCMs under dynamic (30 rpm) and static culture conditions over time. (FIG. 10C) The q-RT PCR analysis showing mRNA expression of pluripotency markers KLF4 and NANOG in hiPSCs cultured in HMCs and 2D culture conditions. (FIG. 10D) The immunostaining of hiPSCs cultured in HMCs and on 2D glass surface against the pluripotency marker OCT4 (magenta). (scale bar: 50 μm; inset scale bar: 20 μm). The differentiation of hiPSCs to cardiomyocytes in HCMs and on 2D glass surface as shown by (FIG. 10E) the mRNA expression levels of cardiomyocyte markers NKX2.5 and troponin-T (TNNT), and (FIG. 10F) the immunostaining against cardiomyocyte marker TNNT. (scale bar: 100 μm; inset scale bar: 20 μm). (N.S. indicates no significance, p>0.05).

FIG. 11 depicts bright and dark field microscope images of HMCs fabricated using various designs. (scale bars: 300 μm)

FIG. 12A and FIG. 12B depict the results of thermally actuating PDMS-composite films. (FIG. 12A) ANSYS simulation of the ROC of the PDMS-composite films at different temperatures. (FIG. 12B) A strip of the developed PDMS-composite film at 7.9° C. (left) and 38.9° C. (right). Thermal actuation can be used to open HMCs to facilitate cell harvesting. (scale bar: 1 mm).

### DETAILED DESCRIPTION

The present invention provides hollow microcarriers for cell culture. The hollow microcarriers form a shell around a hollow interior and can be opened to permit cell infiltration or harvesting. The hollow microcarriers protect cells from hydrodynamic shear stress without hindering the diffusion of nutrients in and out of their hollow interior.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation associated proteins in that cell. When a cell is said to be “differentiating,” as that term is used herein, the cell is in the process of being differentiated.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

The term “derived from” is used herein to mean to originate from a specified source.

“Expandability” is used herein to refer to the capacity of a cell to proliferate, for example, to expand in number or in the case of a cell population to undergo population doublings.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein “growth factors” is intended the following non-limiting factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein at concentrations of between picogram/ml to milligram/ml levels.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which naturally accompany the isolated cell in a tissue or mammal.

As used herein, the term “multipotential” or “multipotentiality” is meant to refer to the capability of a stem cell to differentiate into more than one type of cell.

As used herein, a “pluripotent cell” defines a less differentiated cell that can give rise to at least two distinct (genotypically and/or phenotypically) further differentiated progeny cells.

The terms “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew itself or to produce progeny cells which will differentiate into the desired cell type. Unlike pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

“Progression of or through the cell cycle” is used herein to refer to the process by which a cell prepares for and/or enters mitosis and/or meiosis. Progression through the cell cycle includes progression through the G1 phase, the S phase, the G2 phase, and the M-phase.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

### Hollow Microcarrier

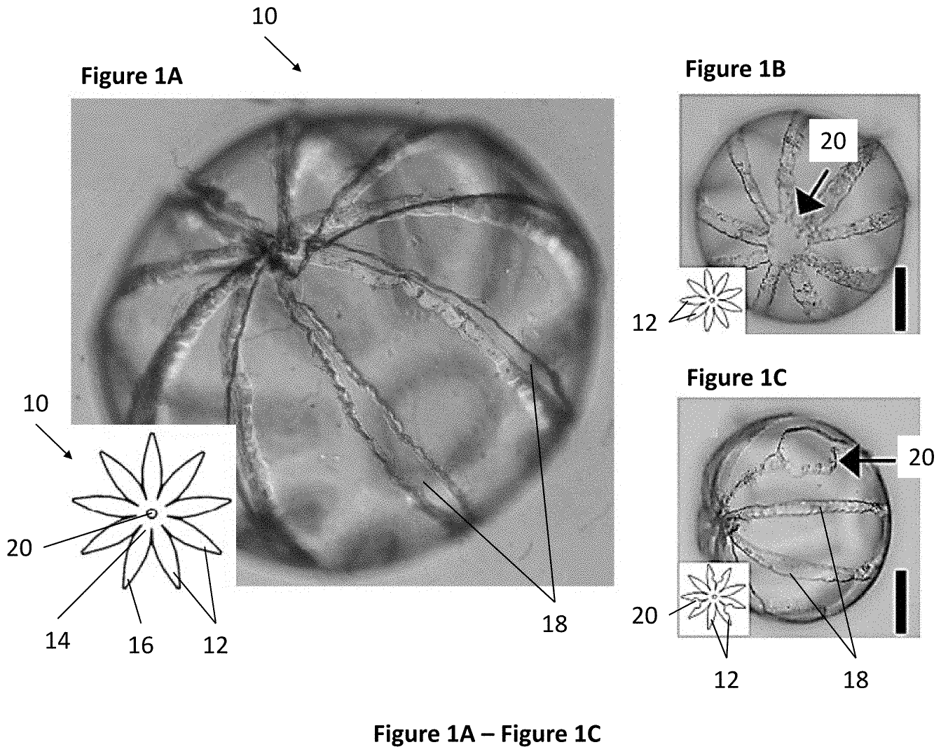

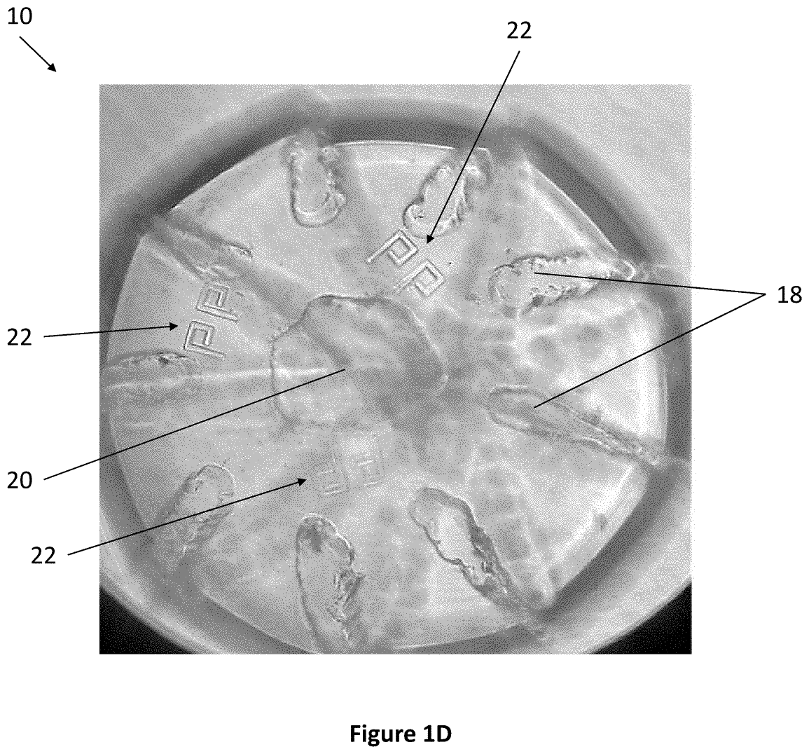

Referring now to FIG. 1A through FIG. 1D, an exemplary hollow microcarrier (HMC) 10 is depicted. HMC 10 comprises a plurality of leaflets 12, each leaflet 12 having a proximal end 14 and a distal end 16. Each leaflet 12 is joined to each other at their proximal ends 14 to form a snowflake or flower-like structure of leaflets 12 arranged in a radial pattern. HMC 10 can have any suitable number of leaflets 12, including 1, 2, 3, 4, 5, 6, 7, 8, 9, 10, or more. The plurality of leaflets 12 can be the same size and length or different sizes and lengths.

HMC 10 comprises an open configuration and a closed configuration. In the open configuration, leaflets 12 are spread apart to give HMC 10 a substantially flat shape. In the closed configuration, leaflets 12 curl towards each other to bring the proximal end 14 of each leaflet 12 together, giving HMC 10 a substantially spherical shape. The closed configuration thereby forms a hollow space within the curled leaflets 12.

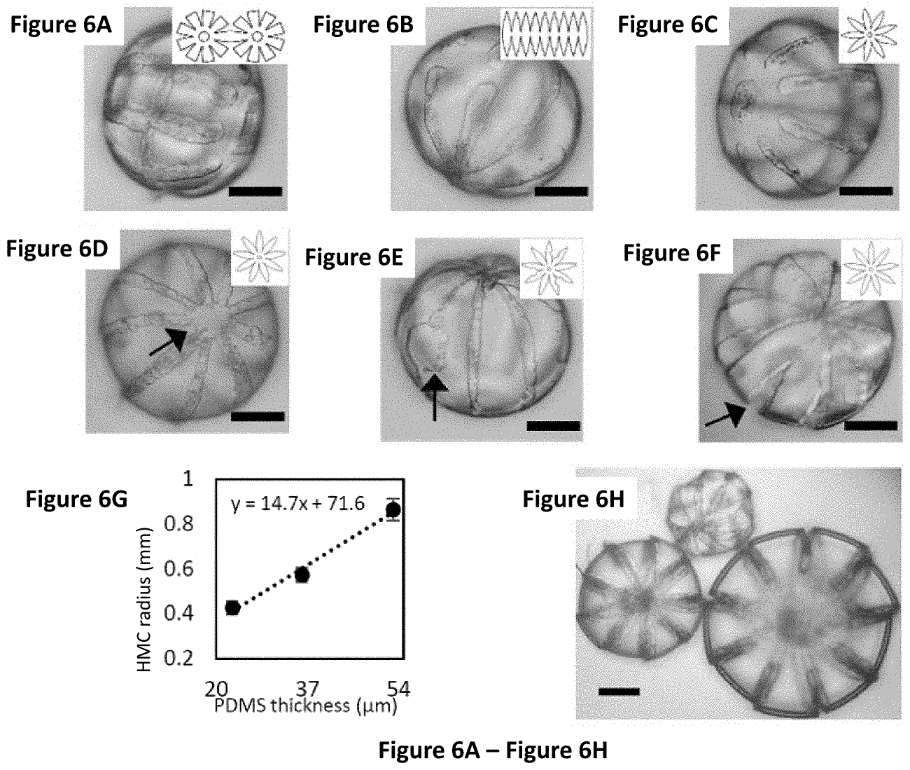

While HMC 10 is depicted in FIG. 1A through FIG. 1D as having a substantially snowflake or flower-like shape in an open configuration and a substantially spherical shape in a closed configuration, it should be understood that HMC 10 can have any suitable shape in the open and closed configurations. The open configurations can include any number of leaflets having any suitable shape or size linked together in any arrangement to form a three-dimensionally shaped closed configuration. For example, the open configuration can include two linked snowflake or flower-like shapes, wherein each snowflake or flower-like shape is constructed from a plurality of leaflets joined in a radial pattern and defines a hemisphere of a substantially spherical or spheroid closed configuration (FIG. 6A). In another example, the open configuration can include a plurality of leaflets, each leaflet defining a gore segment having opposing ends and a central region, wherein each leaflet is joined to an adjacent leaflet at the central region in a linear array to define a substantially spherical or spheroid closed configuration (FIG. 6B). The substantially spherical or spheroid closed configurations can be elongated or flattened in any desired manner. In another example, the open configuration can include a rectangular leaflet linked to two circular leaflets, wherein the leaflets define a substantially cylindrical closed configuration. In another example, the open configuration can include several polygonal leaflets linked together, wherein the polygonal leaflets define a substantially polyhedral closed configuration. The polyhedral closed configuration can include, but is not limited to: tetrahedrons, hexahedrons, octahedrons, dodecahedrons, icosahedrons, and the like. Persons having skill in the art will recognize that by the preceding examples, the present invention should be understood to encompass all HMCs having any open configurations that curl or fold into any three-dimensional shaped closed configurations defining a hollow interior.

In various embodiments, HMC 10 can be modified to provide greater or less access to the hollow interior of its closed configuration. For example, HMC 10 can include narrow leaflets 12, such that in a closed configuration, HMC 10 comprises larger gaps 18 between each adjacent leaflet 12. In another example, HMC 10 can include one or more aperture 20. In some embodiments, an aperture 20 can be provided at the junction where the proximal ends 14 of each leaflet 12 are joined (FIG. 1A). In some embodiments, an aperture 20 can be formed by truncating distal ends 16 of each leaflet 12 (FIG. 1B). In some embodiments, an aperture 20 can be formed within a leaflet 12, or formed between two adjacent leaflets 12 (FIG. 1C).

In various embodiments, HMC 10 can include one or more markings 22 (FIG. 1D). The one or more markings 22 can include, but is not limited to: letters, numbers, shapes, symbols, barcodes, Quick Response (QR) codes, images, and the like. In some embodiments, the one or more markings 22 can be etched or printed onto an outer surface and/or an inner surface of HMC 10, etched as an aperture through the outer surface and inner surface of HMC 10, or embedded between the outer surface and inner surface of HMC 10. In some embodiments, the one or more markings 22 can be formed from an ink, a label, or other physical media. The ink, label, or other physical media can be detectible using an exterior source of light (e.g., visible light and ultraviolet light), or by an internal source of energy (e.g., luminescence and radiation).

HMC 10 can have any suitable size. For example, in some embodiments, HMC 10 can have a closed configuration having a diameter that is typical of bioreactor microcarriers, such as in the range of between about 100 μm and 500 μm. However, HMC 10 is advantageous over traditional microcarriers due to their superior diffusion characteristics and protection from shear stress, thereby allowing the diameter of the closed configuration to be any desired size, such as in the range of between about 10 μm and 10 cm. In certain embodiments, the diameter can be between about 50 μm and 10 mm.

In some embodiments, HMC 10 is constructed from a bilayer of two materials under different levels of stress. Binding a layer of a first material having a first level of stress with a layer of a second material having a second level of stress induces curling in leaflets 12 when the layers are relaxed to form a closed configuration in HMC 10. For example, an HMC 10 comprising a layer of a first material that is under compressive stress bonded to a layer of a second material can have a closed configuration when the layer of the first material is relaxed, wherein the layer of the second material is positioned in the hollow interior of the closed configuration. In another example, an HMC 10 comprising a layer of a first material that is under tensile stress bonded to a layer of a second material can have a closed configuration when the layer of the first material is relaxed, wherein the layer of the first material is positioned in the hollow interior of the closed configuration. Differing levels of tensile stress or compressive stress between the layer of the first material and the layer of the second material can be achieved using any suitable means. In some embodiments, the layer of the first material and the layer of the second material can be fabricated at different processing temperatures to generate the differing levels of tensile stress or compressive stress.

In some embodiments, the layer of first material and the layer of second material can have different swelling ratios. Binding a layer of a first material having a first swelling ratio with a layer of a second material having a second swelling ratio induces curling in leaflets 12 upon immersion in an aqueous solution to form a closed configuration in HMC 10. For example, an HMC 10 comprising a layer of a first material having a higher swelling ratio than a layer of a second material can have an open configuration when damp or dry and a closed configuration when immersed in an aqueous solution, wherein the layer of the second material having the lower swelling ratio is positioned in the hollow interior of the closed configuration. In some embodiments, the open or closed configuration can be controlled by changing the properties of the aqueous solution. For example, the degree of swelling of the layer of the first material, the layer of the second material, or both, can be controlled by adding or removing a concentration of a solute in the aqueous solution (such as a salt) or by changing the solvent.

In some embodiments, the layer of first material and the layer of second material can have different coefficients of thermal expansion. Binding a layer of a first material having a first coefficient of thermal expansion with a layer of a second material having a second coefficient of thermal expansion induces curling in leaflets 12 at certain temperatures to form a closed configuration in HMC 10. For example, an HMC 10 comprising a layer of a first material having a higher coefficient of thermal expansion than a layer of a second material can have an open configuration at a first temperature and a closed configuration at a second temperature, wherein the layer of the second material having the lower coefficient of thermal expansion is positioned in the hollow interior of the closed configuration.

### Methods of Fabricating the Hollow Microcarriers

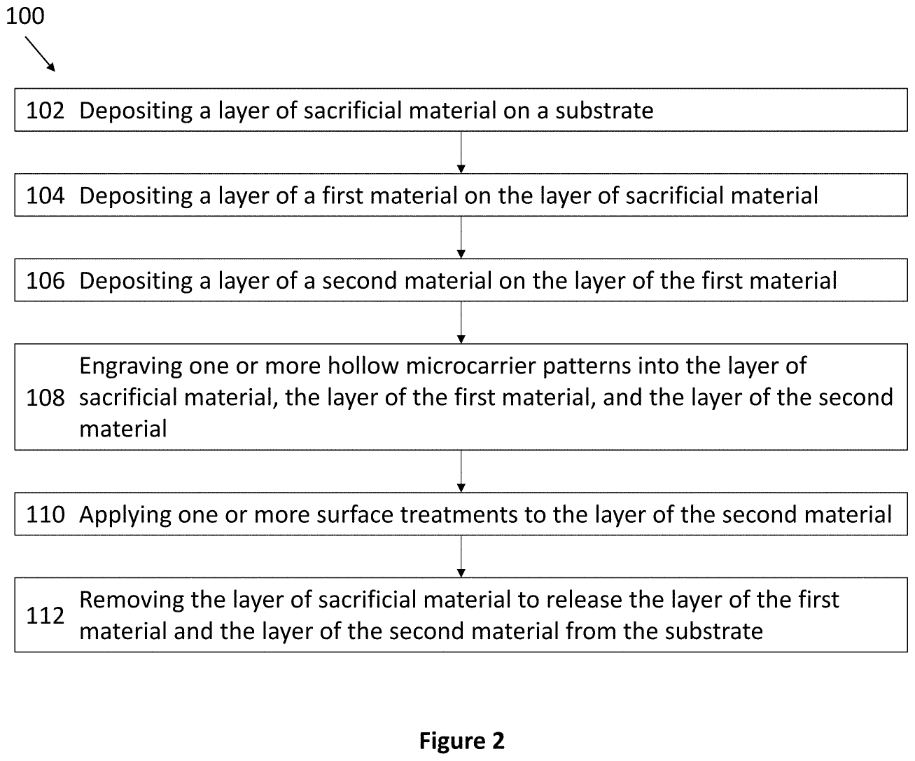

The present invention also relates to methods of fabricating HMCs. Referring now to FIG. 2, an exemplary method 100 of fabricating an HMC is depicted. Method 100 begins with step 102, wherein a layer of sacrificial material is deposited on a substrate. In step 104, a layer of a first material is deposited on the layer of sacrificial material. In step 106, a layer of a second material is deposited on the layer of the first material. In step 108, one or more HMC patterns are engraved into the layer of sacrificial material, the layer of the first material, and the layer of the second material. In step 110, one or more surface treatments are applied to the layer of the second material. In step 112, the layer of sacrificial material is removed to release the layer of first material and the layer of the second material from the substrate, whereupon the layer of the first material and the layer of the second material deform at different degrees to curl into a hollow spherical structure.

In certain embodiments, the methods of fabricating HMCs can include steps for applying one or more markings. As described elsewhere herein, the HMCs can include one or more markings that can be etched or printed onto an outer surface and/or an inner surface of HMC 10, etched as an aperture through the outer surface and inner surface of HMC 10, or embedded between the outer surface and inner surface of HMC 10. The one or more markings can be applied using any suitable method, including photolithography, stereolithography, laser etching, and the like. For example, step 102 can be followed by a step of etching one or more markings into the sacrificial material, such that in step 104, the first material fills in the etching, and in step 112, the layer of sacrificial material is removed to reveal embossed markings on the first material. In another example, step 104 can be followed by a step of printing one or more markings onto the first material, such that in step 106, the second material covers the one or more markings, embedding them between the first material and the second material. In another example, step 108 can include engraving one or more markings into the second material. In another example, step 108 can include engraving one or more markings through the second material, the first material, and the sacrificial material.

The various layers, coatings, and surface treatments described above can be deposited or applied using any suitable means, including spin coating, dip coating, chemical vapor deposition, chemical solution deposition, physical vapor deposition, liquid bath immersion, and the like. The layers, coatings, and surface treatments can be deposited or applied with any suitable thickness. In some embodiments, the thickness of a layer affects the geometry of the fabricated HMC. For example, in certain embodiments, a thinner layer of the first material and/or second material leads to sharper curling of leaflets and enables the fabrication of smaller diameter HMCs, while a thicker layer of the first material and/or the second material leads to more gradual curling of leaflets and enables the fabrication of larger diameter HMCs.

The sacrificial material can be any suitable material that can withstand any high temperature treatments, while also can be easily removed to release the various layers from the underlying substrate. In some embodiments, the sacrificial material is AZ-9260 photoresist spin-coated on a substrate at 1300 rpm for 10 seconds to achieve a layer thickness of 13 μm and baked at 140° C. for 1 hour. AZ-9260 photoresist can be easily removed in an ethanol bath. Likewise, the substrate can be any suitable substrate that is substantially flat and can withstand any high temperature treatments. In some embodiments, the substrate is a silicon wafer.

As described elsewhere herein, the first material and the second material can be any suitable material having different coefficients of thermal expansion, or any suitable material under different levels of stress. In some embodiments, the first and second materials can be selected from a metal, including but not limited to: nickel, titanium, nitinol, gold, silver, copper, platinum, and the like. In some embodiments, the first and second materials can be selected from a polymer, including but not limited to: poly(urethanes), poly(siloxanes) or silicones, poly(ethylene), poly(vinyl pyrrolidone), poly(-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, and the like. In some embodiments, the first material is a mix of Sylgard 184 and Sylgard 3-6636 in a 5:1:3:3 ratio of Base _{Sylgard 184}: Curing agent _{Sylgard 184}: Part-A _{Sylgard 3-6636}: Part-B _{Sylgard 3-6636} that is spin-coated on the sacrificial material at 2000 rpm for 3 minutes to achieve a layer thickness of 18 μm and baked at 40° C. for 12 hours. In some embodiments, the second material is a mix of Sylgard 184 and Xiameter-200M in a 4:1:1 ratio of Base _{Sylgard 184}: Curing agent _{Sylgard 184}: Xiameter that is spin-coated on the first material at 1300 rpm for 2 minutes to achieve a layer thickness of 19 μm and baked at 130° C. for 3 hours.

As described elsewhere herein, one or more surface treatments can be applied to the HMC layers. In some embodiments, the application of the one or more surface treatments can facilitate the application of successive surface treatments. For example, in some embodiments, the one or more surface treatment is a corona discharge treatment to render portions of the HMC layers hydrophilic or hydrophobic. In other embodiments, the one or more surface treatment can include a coating to prevent cell adhesion, such as 2-[Methoxy(Polyethyleneoxy)6-9Propyl]Trimethoxysilane (MPEGTMS). A surface treatment that prevents cell adhesion may be advantageous to on the outer layer of the HMCs. In some embodiments, the application of the one or more surface treatments can facilitate the adherence and growth of cell lines. For example, in some embodiments, the surface treatments include a layer of aminopropyltriethoxysilane (APTES), a layer of glutaraldehyde (GA), and a layer of collagen. In other embodiments, the surface treatments include a layer of Geltrex.

In various embodiments, the one or more surface treatments can include one or more extracellular matrix material and/or blends of naturally occurring extracellular matrix material, including but not limited to collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. In various embodiments, the one or more surface treatments can include one or more carbohydrates such as chitin, chitosan, alginic acids, and alginates such as calcium alginate and sodium alginate. These materials may be isolated from plant products, humans or other organisms or cells or synthetically manufactured.

In various embodiments, the surface treatments can include natural peptides, such as glycyl-arginyl-glycyl-aspartyl-serine (GRGDS), arginylglycylaspartic acid (RGD), and amelogenin. In some embodiments, the surface treatments can include sucrose, fructose, cellulose, or mannitol. In some embodiments, the surface treatments can include nutrients, such as bovine serum albumin. In some embodiments, the surface treatments can include vitamins, such as vitamin B2, vitamin Ad, Vitamin D, Vitamin E, and Vitamin K. In some embodiments, the surface treatments can include nucleic acids, such as mRNA and DNA. In some embodiments, the surface treatments can include natural or synthetic steroids and hormones, such as dexamethasone, hydrocortisone, estrogens, and its derivatives. In some embodiments, the surface treatments can include growth factors, such as fibroblast growth factor (FGF), transforming growth factor beta (TGF-β), and epidermal growth factor (EGF). In some embodiments, the surface treatments can include a delivery vehicle, such as nanoparticles, microparticles, liposomes, viral and non-viral transfection systems.

In various embodiments, the surface treatments can include one or more therapeutics. The therapeutics can be natural or synthetic drugs, including but not limited to: analgesics, anesthetics, antifungals, antibiotics, anti-inflammatories, nonsteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents.

### Methods of Using the Hollow Microcarriers

The present invention also relates to methods of culturing cells using HMCs. Referring now to FIG. 3, an exemplary method 200 of culturing cells using HMCs is depicted. Method 200 begins with step 202, wherein an amount of HMCs is added to a suspension of cells. In step 204, the HMCs are shifted between a closed configuration, an open configuration, and back to a closed configuration in the suspension of cells to introduce cells into the hollow interior of the HMCs. In step 206, the HMCs are incubated under static conditions to provide the cells an opportunity to adhere to the hollow interior of the HMCs. In step 208, the HMCs are incubated under dynamic conditions, such as in a bioreactor. In step 210, the HMCs are shifted from a closed configuration to an open configuration to harvest the cells from the HMCs.

As described elsewhere herein, the HMCs of the present invention have an open configuration and a closed configuration, wherein the plurality of the leaflets of the HMCs are uncurled in an open configuration and are curled in a closed configuration to form a hollow enclosed space. In some embodiments, the HMCs can be shifted between an open configuration and a closed configuration using temperature. For example, HMCs comprising a layer of first material having a different coefficient of thermal expansion than a layer of second material can be shifted into an open configuration at a low temperature, such as between about 1 and 25° C., and can be shifted into a closed configuration at a high temperature, such as between about 25° C. and 40° C. In this manner, the HMCs will remain closed under high temperature culture conditions, but can be opened for cell seeding or harvesting in a user-controlled lower temperature environment.

In other embodiments, the HMCs can be shifted between an open configuration and a closed configuration mechanically. For example, HMCs comprising a layer of first material under a different level of stress than a layer of second material can be temporarily and reversibly deformed from a closed configuration into an open configuration using a mechanical force, and upon removal of the mechanical force, the HMCs return to a closed configuration. In some embodiments, the mechanical force can be applied by passing the HMCs through a small orifice, such as a pipette tip. Squeezing through the orifice causes the HMCs to deform slightly, widening the gaps between leaflets and allowing cells to infiltrate or exit the hollow interior of the HMCs. After exiting the orifice, the HMCs return to their original closed configuration.

The cells that can be cultured using the HMCs of the present invention can be any suitable cell. For example, in some embodiments the cells can include progenitor cells, pluripotent cells, stem cells, other differentiable cells, and the like. In some embodiments, the HMCs of the present invention direct differentiation of progenitor cells and/or stem cells. In some embodiments, the HMCs of the present invention direct and maintain phenotype plasticity of the cells that are seeded therein. In some embodiments, the HMCs of the present invention are used to support niche expansion of stem cells seeded therein. In some embodiments, the HMCs of the present invention can be used to culture recombinant cells to produce biopharmaceutical products, including therapeutic proteins and monoclonal antibodies.

In various embodiments, the HMCs of the present invention can be used in combination with one or more bioreactors in order to support the expansion and differentiation of cells seeded in the HMCs. In some embodiments, the HMCs can be used in combination with any suitable bioreactor, such as microcarrier-based stirred bioreactors, packed-bed bioreactors, fluidized-bed bioreactors, hollow fiber bioreactors, simulated microgravity bioreactors such as high aspect ratio vessel bioreactors, and slow turning lateral vessel bioreactors.

Typical bioreactors utilize a chamber filled with media, such as DMEM supplemented with 10% (v/v) newborn calf serum (NBCS, 16010159, Life Technologies, USA) and 1% (v/v) penicillin streptomycin (15140122, Life Technologies, USA), and vented to ensure that there is a zero-head space in the reactor chamber. In some embodiments, the reactor chamber is then incubated at 37° C. and the media pumped through a media gas exchange module having its gas exchange tubing filled with a gas mixture of 5% CO₂, 5% 02, and 90% N₂.

In some embodiments, the bioreactor cell culture system is scalable for commercial production of viable cells. In some embodiments, the bioreactors of the present invention have been optimized for the expansion of stem cells, for example human fibroblast derived hiPSCs. The derived hiPSCs may be initially cultured from frozen stocks in a tissue culture flask, trypsinized, and seeded onto the HMCs of the present invention. In some embodiments, the seeded HMCs are then introduced into the bioreactor chamber through a port in an end cap or in the wall of the sleeve of an exemplary bioreactor. Typically, the hiPSCs and/or seeded HMCs are introduced into the bioreactor chamber through a sampling port. In some embodiments, the vessel is slowly rotated without media flow for 24 hours to allow an opportunity for the hiPSCs to efficiently seed the HMCs. In a suspension culture, hiPSCs may readily attach to the HMCs at rotational speeds between 1-7 rpm. After 24 hours, the media flow may be initiated and a sample of the media in the culture chamber may be taken and the number of unattached cells counted to assess the seeding efficiency to the HMCs.

In various embodiments, methods and compositions of a defined media which supports stem cell self-renewal are described herein. A benefit of using a defined media is that the ingredients comprising the media are known and have known quantities. In contrast, an undefined medium has some complex ingredients, consisting of a mixture of many, many chemical species in unknown proportions. The reasons for utilizing chemically defined media are also pragmatic because such media is reproducible at different times and in different laboratories. Defined media can be varied in a controlled manner and are free of unknown biological activities, such as enzymes and, alternatively, growth factors, which may affect the responses being studied.

In some embodiments, the compositions and methods useful with the present invention enhance the culturing of cells, for example, differentiable cells such as embryonic stems cells, hematopoietic stem cells, adipose derived stem cells, bone marrow derived stem cells and the like. In some embodiments, the differentiatable cells are directed to differentiate into cells of target tissues, for example fibroblasts, osteocytes, epithelial cells, endothelial cells, myocytes, neurocytes, and the like. In some embodiments, at different points during culturing the differentiable cells, various components may be added to the cell culture such that the medium can contain components such as growth factors, differentiation factors, and the like other than those described herein.

In some embodiments, the compositions and methods can comprise a basal salt nutrient solution. A basal salt nutrient solution refers to a mixture of salts that provide cells with water and certain bulk inorganic ions essential for normal cell metabolism, maintain intra- and extra-cellular osmotic balance, provide a carbohydrate as an energy source, and provide a buffering system to maintain the medium within the physiological pH range. For example, basal salt nutrient solutions may include, but are not limited to, Dulbecco's Modified Eagle's Medium (DMEM), Minimal Essential Medium (MEM), Basal Medium Eagle (BME), RPM1 1640, Hams F-10, Ham's F-12, (β- Minimal Essential Medium (β-MEM), Glasgow's Minimal Essential Medium (G-MEM), and Iscove's Modified Dulbecco's Medium, and mixtures thereof. In some embodiments, the basal salt nutrient solution is an approximately 50:50 mixture of DMEM and Ham's F12.

In some embodiments, the compositions and methods useful with the present invention provide for one or more soluble attachment factors or agents, such as soluble attachment components as contained in the human serum, which at the appropriate concentration range facilitates cell attachment to tissue culture type plastic and or the HMC surface. Such cell attachment allows cells to attach and form a monolayer but in the absence of a feeder layer or a substrate coating, e.g., a matrix coating, Matrigel, and the like. In some embodiments, human serum is utilized in order to provide an animal-free environment. In some embodiments, serum from animal sources, for example goat, calf, bovine, horse, mouse, and the like is utilized. Serum can be obtained from any commercial supplier of tissue culture products, examples include Gibco-Invitrogen Corporation (Grand Island, N.Y. USA), Sigma (St. Louis Mo., USA) and the ATCC (Manassas, Va. USA). The serum used may be provided at a concentration range of about 0.1% to about 20%, about 5% to about 15%, about 7% to about 12%, about 10%, 0.1 to about 3%, about 0.5 to about 2%, about 0.5 to about 1.5%, and about 0.5 to about 1%.

In some embodiments, the defined culture conditions may comprise proliferating pluripotent stem cells substantially free of feeder cells or layers, or “feeder-free”, or a conditioned medium produced by collecting medium from a culture of feeder cells. In some embodiments, differentiable cells are contacted with at least one composition in the absence a feeder cell layer, such that the cells are maintained in an undifferentiated state for at least one (1) to twelve (12) months or more. Pluripotency can be determined through characterization of the cells with respect to surface markers, transcriptional markers, karyotype, and ability to differentiate to cells of the three germ layers. These characteristics are well known to those of ordinary skill in the art.

In some embodiments, as contemplated herein, the differentiable cells can be passaged using enzymatic, non-enzymatic, or manual dissociation methods prior to and/or after contact with a defined medium. Non-limiting examples of enzymatic dissociation methods include the use of proteases such as trypsin, collagenase, dispase, and accutase (marine-origin enzyme with proteolytic and collagenolytic enzymes in phosphate buffered saline; Life Technologies, Carlsbad, Calif). In some embodiments, accutase is used to passage the contacted cells. When enzymatic passaging methods are used, the resultant culture can comprise a mixture of singlets, doublets, triplets, and clumps of cells that vary in size depending on the enzyme used. A non-limiting example of a non-enzymatic dissociation method is a cell dispersal buffer. Manual passaging techniques have been well described in the art, such as in Schulz et al., 2004 Stem Cells, 22(7):1218-38. The choice of passaging method is influenced by other culture conditions, including but not limited to feeders and/or extracellular matrices.

In some embodiments, the methods described herein allow for expansion of human stem cells, followed by dissociation of aggregates and passaging of the disassociated cells so that the cells retain their pluripotency through expansion and serial passages. In addition, the methods of expansion and passage described herein are carried out in a closed system which ensures sterility during the production process.

Methods of inducing differentiation are known in the art and can be employed to induce the desired stem cells to give rise to cells having a mesodermal, ectodermal or endodermal lineage.

After culturing the stem cells in a differentiating-inducing medium for a suitable time (e.g., several days to a week or more), the stem cells can be assayed to determine whether, in fact, they have acquired the desired lineage.

Methods to characterize differentiated cells that develop from the stem cells of the invention, include, but are not limited to, histological, morphological, biochemical and immunohistochemical methods, or using cell surface markers, or genetically or molecularly, or by identifying factors secreted by the differentiated cell, and by the inductive qualities of the differentiated stem cells.

In another embodiment, the cells can be genetically modified, e.g., to express exogenous (e.g., introduced) genes (“transgenes”) or to repress the expression of endogenous genes, and the invention provides a method of genetically modifying such cells and populations. In accordance with this method, the cells are exposed to a gene transfer vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA or a ribozyme).

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). Of course, the choice of vector will largely determine the method used to introduce the vector into the cells (e.g., by protoplast fusion, calcium-phosphate precipitation, gene gun, electroporation, infection with viral vectors, etc.), which are generally known in the art.

The genetically altered cells can be employed to produce the product of the transgene. In other embodiments, the genetically modified cells are employed to deliver the transgene and its product to an animal. For example, the cells, once genetically modified, can be introduced into the animal under conditions sufficient for the transgene to be expressed in vivo.

In other embodiments, cells can be employed as therapeutic agents, for example in cell therapy applications. Generally, such methods involve transferring the cells to desired tissue, either in vitro (e.g., as a graft prior to implantation or engrafting) or in vivo, to animal tissue directly. The cells can be transferred to the desired tissue by any method appropriate, which generally will vary according to the tissue type. For example, cells can be transferred to a graft by bathing the graft (or infusing it) with culture medium containing the cells. Alternatively, the cells can be seeded onto the desired site within the tissue to establish a population. Cells can be transferred to sites in vivo using devices such as catheters, trocars, cannulae, stents (which can be seeded with the cells), etc.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: Hollow Microcarriers for Large-Scale Expansion of Anchorage-Dependent Cells in a Stirred Bioreactor

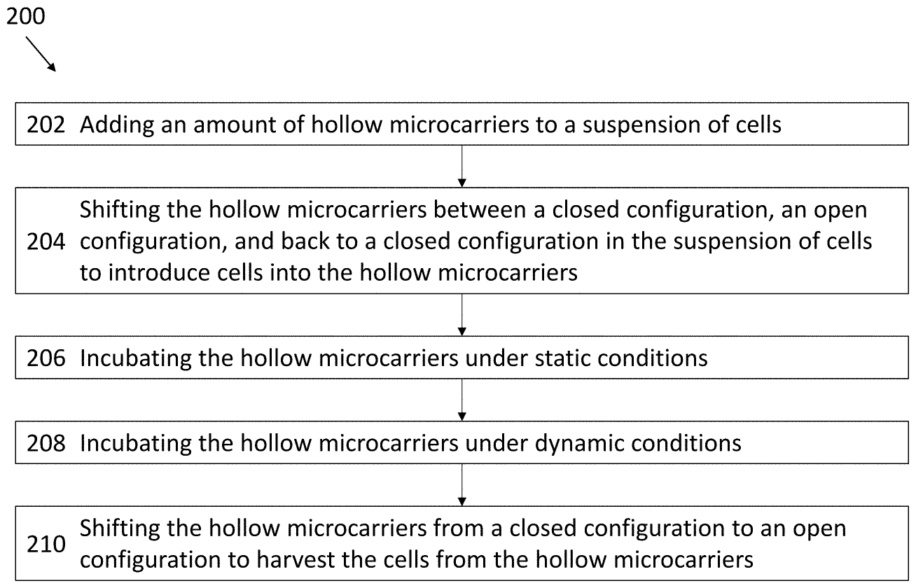

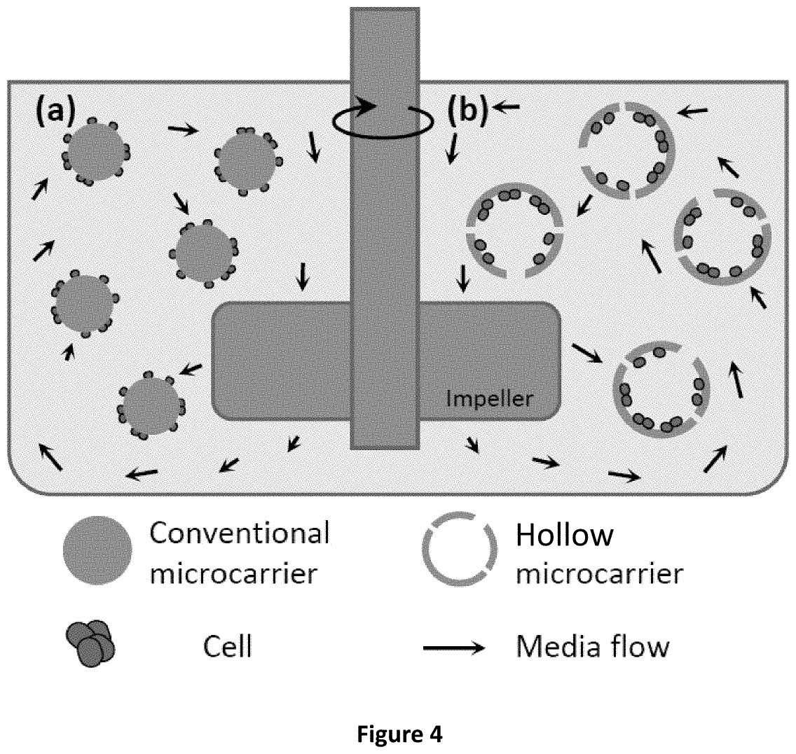

The following study demonstrates hollow microcarriers (HMCs) as a viable alternative to conventional microcarriers. HMCs are microspheres with hollow interiors that permit target cell attachment and culture, as shown in FIG. 4. Unlike conventional microcarriers, which directly expose cells to external turbulent flow, HMCs protect the cells from the hydrodynamic shear stress. Openings in HMCs provide sufficient nutrients to the cells within. The fabrication process and numerical analysis of HMCs are presented, followed by the expansion of NIH/3T3 fibroblasts with HMCs. The study also demonstrates the expansion and cardiac differentiation of human induced pluripotent stem cells (hiPSC).

The materials and methods are now described.

### Fabrication of Pre-Stressed PDMS Bilayer Film

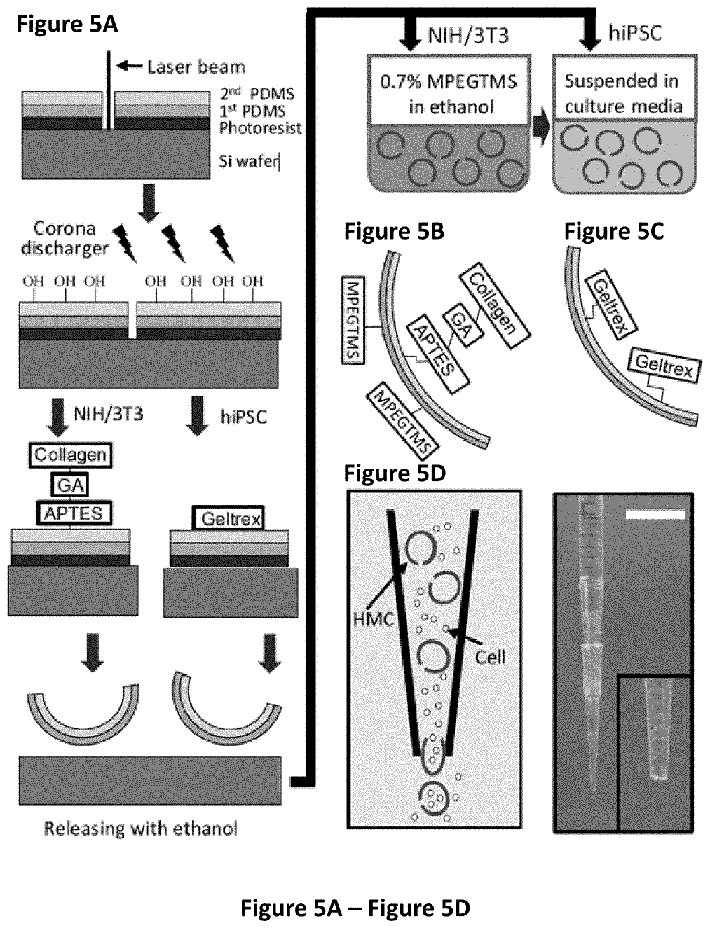

The fabrication process is presented in FIG. 5A through FIG. 5D. A thick positive photoresist (AZ-9260, AZ Electronic Materials, USA) was spin-coated on a 4-inch silicon wafer at 1300 rpm for 10 seconds with a target thickness of 13 μm and baked for 1 hour at 140° C. Sylgard 184 (01064291, Dow Corning, USA) and Sylgard 3-6636 (01901443, Dow Corning, USA) were mixed with a mixing ratio of 5:1:3:3 (Base _{Sylgard 184}: Curing agent _{Sylgard 184}: Part-A _{Sylgard 3-6636}: Part-B _{Sylgard 3-6636}). The mixture was degassed in a vacuum chamber and spin-coated on the wafer at 2000 rpm for 3 minute with a target thickness of 18 μm. The wafer was baked at 40° C. overnight. The second layer of PDMS is a mixture of Sylgard 184 and Xiameter-200M (Dow Corning, USA) with a mixing ratio of 4:1:1 (Base _{Sylgard 184}: Curing agent _{Sylgard 184}: Xiameter). The mixture was degassed and spin-coated on the wafer at 1300 rpm for 2 minutes with a target thickness of 19 The wafer was baked on a hotplate at 130° C. for 3 hours.

### Engraving and Surface Treatment

The pattern of HMCs was engraved on the pre-stressed PDMS film using a laser engraver (VLS 2.30, Universal laser system Inc., USA). The engraved film was treated with a corona discharger (BD-20, ElectroTechnic Products, USA) to render it hydrophilic. For NIH/3T3 fibroblasts, the wafer was immersed in an aqueous solution of 1% 3-Aminopropyltriethoxysilane (APTES) (440140, Sigma-Aldrich, USA) and incubated at 37° C. for 1 hour. After washing the wafer with phosphate buffered saline (PBS), the wafer was immersed in an aqueous solution of 0.1% glutaraldehyde (GA) (G5882, Sigma-Aldrich, USA) for 20 minutes at room temperature, followed by rinsing with PBS twice. The film was then coated with collagen (A1048301, Life Technologies, USA). The collagen was diluted with 0.2M acetic acid to 50 μg/ml. The PDMS film was functionalized with the collagen solution at room temperature for 1 hour, followed by a PBS rinse. For hiPSC, APTES and GA treatments were not necessary, as hiPSC cannot attach to unmodified PDMS. Geltrex (A1413201, Life Technologies, USA) was diluted in Dulbecco's modified eagle medium (DMEM, 11995040, Life Technologies, USA) at 1% (v/v) and used to functionalize the PDMS film at room temperature. After 1 hour, the PDMS film was washed with PBS.

### Release Process of HMC

The photoresist layer was dissolved by immersing the wafer in ethanol. After 6 minutes, the patterns bent upward and formed HMCs. The HMCs were collected in a 15 ml tube filled with ethanol. For NIH/3T3 fibroblasts, HMCs were transferred to an ethanol solution of 0.7% (v/v) 2-[Methoxy(Polyethyleneoxy)6-9Propyl]Trimethoxysilane (MPEGTMS) (65994-07-2, Gelest, USA) and kept in room temperature for 15 minutes. MPEGTMS bound only to the outside surface of HMCs and prevented cell attachment, because most of the available OH group on the inner surface were already reacted to APTES. Subsequently, HMCs were washed twice with ethanol. In order to transfer the HMCs to DMEM, the ethanol with HMCs was slowly added on top of a 15 ml tube filled with DMEM to maintain separate layers of the ethanol and the DMEM. The HMCs are heavier than both ethanol and DMEM, causing them to gradually descend to the bottom of the tube after 30 minutes. The ethanol was aspirated and the HMCs were washed with DMEM. For hiPSC, HMCs were rinsed and stored in culture media after releasing them from ethanol. Fully functionalized HMCs for the fibroblasts and the hiPSC are illustrated in FIG. 5B and FIG. 5C.

### Numerical Analysis

ANSYS Workbench (ANSYS, Inc., USA) was used to analyze the shear stress and the glucose concentration of HMC. To calculate the shear stress, a CFX fluid flow module was used and the wall shear was calculated as a measure of the hydrodynamic shear stress on both sides of HMC. The HMC was placed in a cube with a 3 mm edge. The in-flow of 1 m/s was set at two opposite faces of the cubes and the out-flow condition was set at other two opposite faces. The steady-state thermal analysis module was used to calculate the glucose concentration, as the mass diffusion equation is identical to that of the thermal analysis module. The following parameters were employed in the analysis: a cell concentration of 10⁵ cells/cm², a glucose consumption rate of 1 ng/day/cell (Trummer E et al., Biotechnology and bioengineering, 2006, 94(6):1033-1044), a glucose diffusivity of 9.58E-10 m²/s (Haynes WM, ed. CRC handbook of chemistry and physics. CRC press, 2014), and a bulk glucose concentration of 4500 mg/L.

### Cell Culture

Fibroblasts NIH/3T3 were maintained in cell culture flasks in DMEM supplemented with 10% (v/v) newborn calf serum (NBCS, 16010159, Life Technologies, USA) and 1% (v/v) penicillin streptomycin (15140122, Life Technologies, USA). The fibroblasts were subcultured every 5-6 days at 80% confluency. Prior to the experiment, cells were harvested using trypsin (25200056, Life Technologies, USA) and counted.

The human fibroblast derived hiPSCs (DiPS-1016SevA, Harvard stem cell science, USA) were seeded on Geltrex (A1413202, ThermoFisher Scientific, USA) coated tissue culture flasks using mTeSR-1 (05850, StemCell Technologies, Canada) supplemented with 5 μM ROCK inhibitor (Y-27632) (72302, StemCell Technologies, USA) and maintained in mTeSR-1. At around 80% confluency, the hiPSCs were passaged using accutase (07920, StemCell Technologies, Canada).

### HMC Seeding and Culture Protocol

The cell seeding procedure is shown in FIG. 5D. For fibroblast seeding, the collagen coated HMCs were added to a cell suspension of 10⁵ cells/ml. The cell suspension with HMCs was passed through a small orifice of 0.8 mm diameter, which was cut from a 200 μl pipette tip. As the HMCs passed through the orifice, they were temporarily squeezed and recovered to their flattened shape, allowing the cells to enter the HMCs. The seeded HMCs were placed over a cell strainer (10199-658, VWR, USA) in a 6-well plate to separate the cells that were not inside the HMCs. The HMCs were incubated for 6 hours in a static condition to promote cell attachment. Afterward, the HMCs were transferred to a spinner flask (CLS-1430-100, Chemglass, USA) with 100 ml of growth media and cultured under a humidified atmosphere of 5% CO₂ in the incubator at 37° C. The spinner flask was stirred by a slow-speed stirrer (440811, Corning, USA) at 25-180 rpm.

For hiPSC seeding, the hiPSCs were collected using accutase and a cell solution of 10⁶ cells/ml was prepared. HMCs were added to the cell suspension and seeded, following the same procedure as fibroblast seeding. The hiPSC seeded HMCs were then collected and maintained in mTeSR-1 in a static condition (24 well plates) or in a dynamic condition (30 rpm) provided by the same setup used for fibroblasts.

### Proliferation Assay Protocol

The proliferation of the fibroblasts in the HMCs was characterized with Cell Counting Kit-8 (CK04, Dojindo, Kumamonto, Japan) following manufacture's instruction. At the end of 3-hour incubation, the media was transferred into a separate 96-well plate to avoid optical interference of HMCs to the optical measurement. The proliferation of hiPSC in the HMCs were characterized in the static or dynamic culture conditions on days 1, 3, 5, 7, and 10 of culture to determine cell growth, using alamarBlue assay (DAL110, Invitrogen, USA) following manufacturer's instructions. The HMCs with hiPSC were incubated for 6 hours at 37° C., before transferring the media into a separate 96-well plate for measurement. For both cases, calibration curves were used to extract the cell number per HMC from the absorbance values at each time point.

### Cardiomyocyte Differentiation of hiPSCs

Cardiomyocyte differentiation of hiPSCs was adapted from a previously established protocol (Lian X et al., Nature protocols, 2013, 8(1):162). Briefly, differentiation was started by incubating the hiPSCs in RPMI Medium 1640 (11875093, ThermoFisher Scientific, USA) supplemented with B27 without insulin (A1895601, Gibco, USA), beta-mercaptoethanol (M6250, SigmaAldrich, USA) and P/S (1%) (iCM basal media) with the addition of Wnt activator CHIR99021 (CHIR, 10 μM) (04-0004, Stemgent, USA). On day 2, the media of hiPSCs was changed to iCM basal media without any CHIR. On day 4, Wnt inhibitor IWP-2 supplemented iCM basal media (5 μM) (04-0036, Stemgent, USA) was introduced. On day 6, the media was changed to iCM basal media without any small molecules. On day 9 of differentiation, hiPCs were switched to iCM basal medium supplemented with B27 with insulin (17504044, Gibco, USA) and maintained in this medium from day 9-on with media changes every 3 days. Beating was observed on day 12 of differentiation both in HMCs and on 2D surfaces.

### Quantitative Reverse Transcription Polymerase Chain Reaction (q-RT PCR)

Total mRNA was collected from hiPSCs (day 4 after seeding) or iCMs (day 21 of differentiation) cultured in HCMs or on 2D surfaces using an mRNA isolation kit (74104, Qiagen, Netherlands) following manufacturer's instructions. The collected mRNA was then reverse transcribed to cDNA using a cDNA synthesis kit (1708840, Bio Rad, USA) following manufacturer's instructions. The cDNA was then used for the PCR reaction using a real time PCR (1725120, BioRad, USA). The primers for NKX2.5 (assay ID: qHsaCED0001067, BioRad, USA) and cardiac troponin-T (TNNT) (assay ID: qHsaCID0014544, BioRad, USA) were purchased from BioRad. The sequences for NANOG and KLF4 primers were custom made (Eurofins, USA) and the corresponding sequences of KLF4 and NANOG are TATGACCCACACTGCCAGAA (forward) (SEQ ID NO. 1)/TGGGAACTTGACCATGATTG (reverse) (SEQ ID NO. 2) and CAGTCTGGACACTGGCTGAA (forward) (SEQ ID NO. 3)/CTCGCTGATTAGGCTCCAAC (reverse) (SEQ ID NO. 4), respectively.

### Immunostaining

hiPSCs (day 4 after seeding) or iCMs (day 21 of differentiation) cultured in HMCs or on 2D surfaces were fixed using 4% paraformaldehyde (15710, EM Sciences, USA) for 15 minutes at room temperature. The cells were then permeabilized using triton X-100 (85111, ThermoFisher Scientific, USA) (0.1%) for 20 minutes followed by a blocking step with goat serum (G9023, SigmaAlrdich, USA) (10%, 1 h at RT). After the blocking step, the cells were incubated with primary antibodies against OCT-4 (MA1-104, ThermoFisher Scientific, USA) or TNNT (ab45932, Abcam, USA) overnight (1:150 dilution in 10% goat serum, at 4° C.). After washing off excess dye, the cells were incubated with the corresponding secondary antibodies (R37117, and A-11001, ThermoFisher Scientific, USA) for 6 hours (1:200 dilution in 10% goat serum, at 4° C.). The nuclei of the cells were stained with DAPI. The samples were then mounted in anti-fade reagent (P36930, ProLong Gold, Thermo Fisher Scientific, USA) and imaged using a fluorescence microscope (Zeiss Hamamatsu ORCA flash 4.0).

The results are now described.

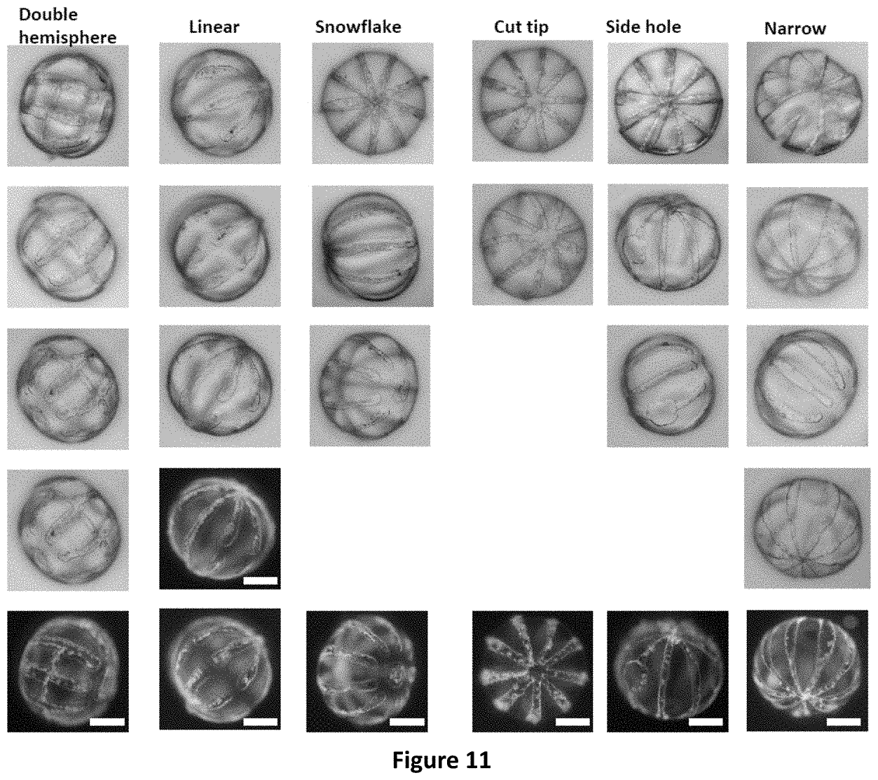

Various geometries of HMCs were produced and tested, as shown in FIG. 6A through FIG. 6H and FIG. 11. For proper seeding, an HMC should be flexible enough to deform during the seeding procedure in order to introduce cells into it. At the same time, it should be rigid enough to protect the cells from the hydrodynamic shear stress in bioreactors. The double hemisphere pattern in FIG. 6A consists of two hemispheres which bend toward each other to form a complete sphere. This design is potentially good for cell seeding; however, it is not adequately rigid. Also, the two hemispheres are connected with one leaflet and has a tendency to tangle or twist in the seeding process. The linear pattern shown in FIG. 6B mitigates some of the shortcomings of the double hemisphere pattern. The linear pattern has a long straight middle section, where the cells can freely migrate for maximum use of the culture area. The middle section also works as a back bone and supports the entire structure. However, the linear pattern is less reliable in forming the hollow sphere. Instead of bending along its length, it sometimes bends in the opposite direction and forms a tube. Lastly, a snowflake pattern is used to produce HMCs as shown in FIG. 6C. It consists of a center area with nine leaflets that bend and lean toward each other to enclose a sphere. The snowflake pattern produces a sufficiently rigid HMC that maintains its form even when it is rotating in the spinner flask. At the same time, it can be squeezed to deform and introduce cells to its hollow interior during the cell seeding process. Because of these advantages, the snowflake pattern was used in this study. In order to fine-tune the performance of the HMCs, the snowflake pattern can be further modified, as shown in FIG. 6D through FIG. 6F. Additional openings can be made to enhance the exchange of media into the HMC by cutting the tip off of the leaflets as shown in FIG. 6D, or by forming side holes as shown in FIG. 6E. Furthermore, the leaflets can be narrowed to adjust the opening gap between the leaflets, as shown in FIG. 6F.

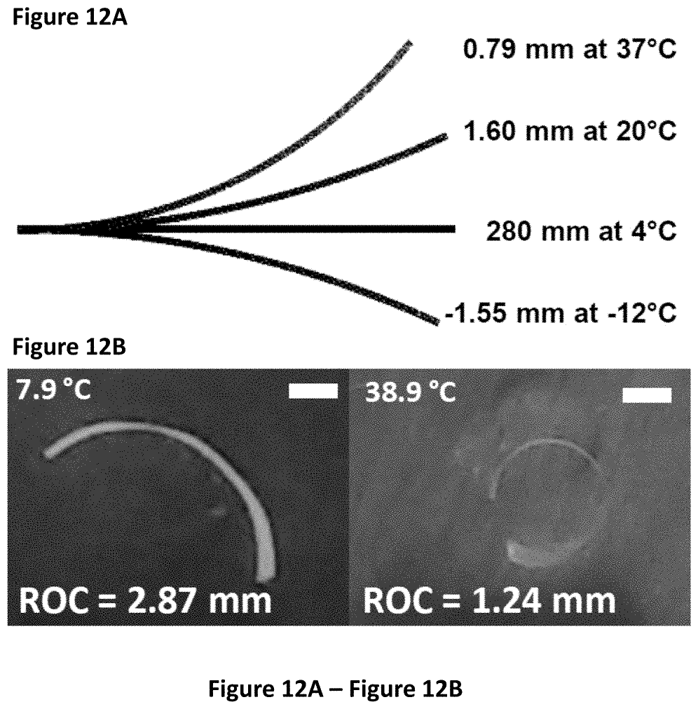

HMCs can be created in different sizes as shown in FIG. 6G and FIG. 6H. During the deposition of the PDMS layers, the thickness of the film was controlled by varying the speed of the spin-coater, which resulted in HMCs with different diameters. The HMCs used in this study have a diameter of 1 mm, which is larger than typical microcarriers. The volume of the HMC was 0.52 ul, whereas the surface area was 3.14 mm², leading to a surface area to volume ratio of 60.4 cm²/ml without considering the volume between HMCs in stirred bioreactors. For comparison, the surface area to volume ratio used with commercial microcarriers is in the range of 8˜80 cm²/ml (G. E. Healthcare and Amersham Biosciences. “Microcarrier cell culture: principles and methods.” General Electric Company (2005)). As shown in FIG. 6G, the diameter of the HMC can be further reduced by reducing the film's thickness or by changing the fabrication conditions. With sufficient miniaturization, the surface area to volume ratio of HMCs can be increased to a level similar to commercial microcarriers. As the size of HMCs decrease, a smaller orifice would be required for deforming the HMCs for seeding cells. This could expose the cells to higher shear stress during the process and potentially damage the cells. To avoid possible damage to the cells, thermal actuation can be used to open the HMCs at lower temperatures, as demonstrated in FIG. 12A and FIG. 12B. In this scheme, the HMCs are opened at low temperature to introduce cells and closed at 37° C. for culture.

For large-scale culture with HMCs, the mass-fabrication of HMCs is essential. For the purposes of the study, HMCs were made from thin PDMS films, which were manually spin-coated and baked on a silicon wafer. Although the fabrication process shown in FIG. 5A through FIG. 5D was able to provide enough HMCs for the current study to demonstrate the feasibility, the process may be adapted for large-scale culture. For example, batch processing with an automated spin-coater and wafer handlers may significantly increase the fabrication throughput and provide sufficient HMCs for billions of cells. Additionally, a roll-to-roll process that bonds and patterns two pre-stressed polymer films could also be used to mass-produce HMCs.

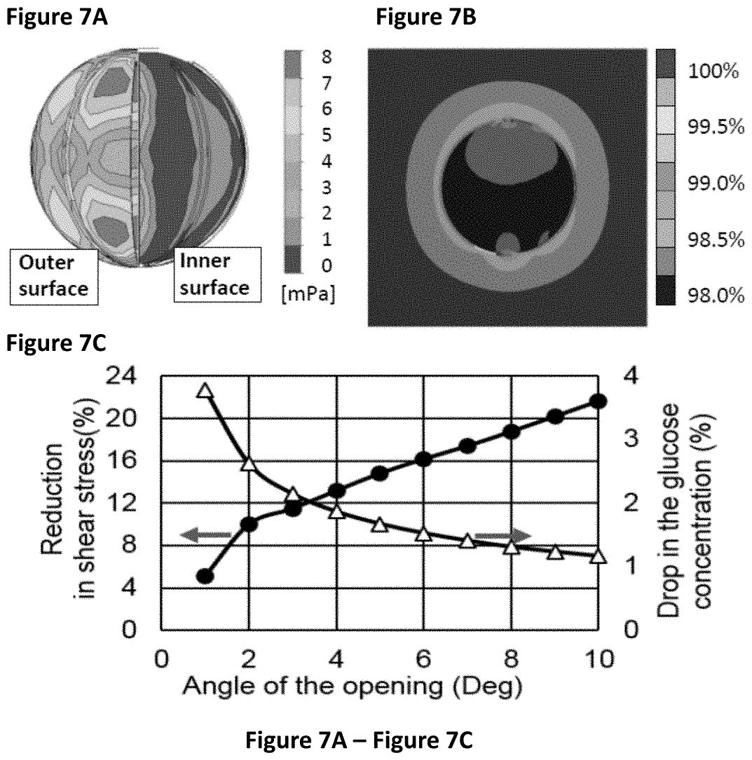

A significant reduction in hydrodynamic shear stress on the inner surface and a negligible drop of glucose concentrations in the HMCs were confirmed with numerical analysis, as shown in FIG. 7A through FIG. 7C. The average shear stress on the outer surface was around 4.71 Pa and on the inner surface was about 1.06 Pa. Although these numbers were based on the assumed media flow velocity as defined in the methods section, this simulation clearly showed that the shear stress is reduced 4 times with the HMCs. The glucose concentration in the HMCs with a confluent cell layer was decreased by less than 2%, as shown in FIG. 7B. The glucose concentration was mostly uniform in the HMCs and a slight increase near the opening was observed. The numerical analysis was based on a fully confluent cell layer and the glucose concentration would be closer to the bulk concentration with a non-confluent cell layer. The shear stress can be further reduced by decreasing the gap between leaflets. However, decreasing the gap will reduce the diffusion of glucose into the HMC and further decrease glucose concentration in the HMC. This tradeoff between the shear stress and the nutrient diffusion can be fine-tuned by varying opening angle or the angle of the gap between leaflets, as shown in FIG. 7C. The HMCs used in FIG. 7A and FIG. 7B had an opening angle of 4°.

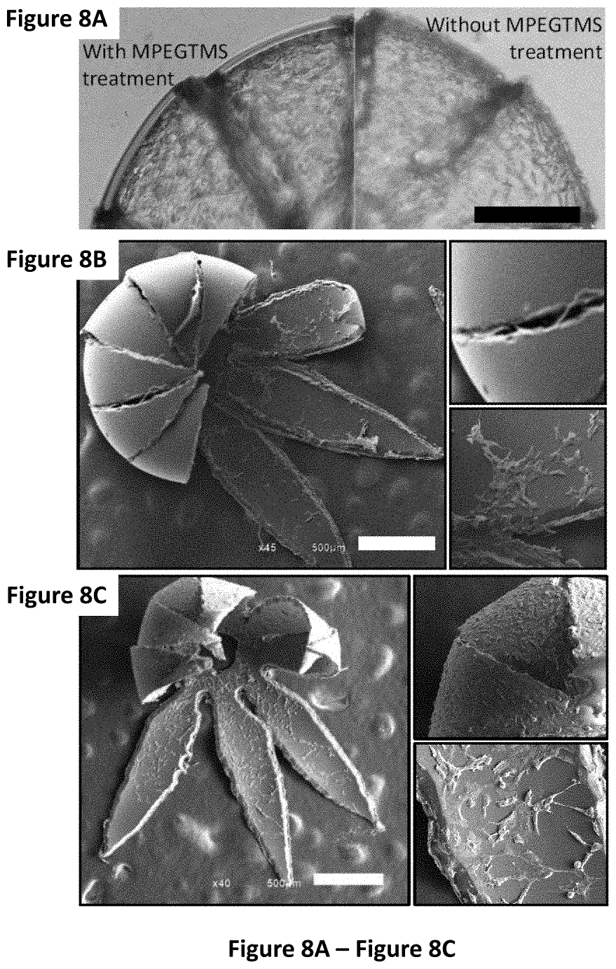

FIG. 8A through FIG. 8C show the effect of MPEGTMS treatment on HMC for NIH/3T3. Unlike hiPSC, NIH/3T3 can potentially attach to unmodified PDMS in culture media. To prevent cell attachment on the outside surface of the HMCs, the inner surface was functionalized with APTES, GA, and collagen, sequentially, followed by immersing the HMCs in an ethanol solution of MPEGTMS. MPEGTMS requires OH-groups to attach to PDMS. Since the available OH groups on the inner surface were already occupied by APTES, MPEGTMS only attached to the outer surface, effectively preventing cell attachment to the outer surfaces. On the other hand, hiPSC cannot attach to a PDMS surface without Geltrex coating. Therefore, additional surface passivation was not necessary and Geltrex coating on the inner surface was sufficient to contain hiPSC in HMCs. FIG. 8A shows the difference between HMCs with and without MPEGTMS treatment. NIH/3T3 were cultured in HMCs for 6 days and phase contrast images were taken. The focus was placed on the outer edge in the middle of the HMCs. The HMCs with treatment had smooth edges, indicating no cells on the outer surface. The untreated HMCs showed rough edges, due to the height of the cells on the outside surface. To further demonstrate the effect of MPEGTMS treatment, HMCs were imaged using scanning electron microscopy (SEM). HMCs seeded with NIH/3T3 were fixed with formaldehyde and dried using hexamethyldisilazane. Prior to platinum sputtering for higher contrast in SEM, a few leaflets of the HMCs were opened manually with tweezers to expose the interior. As shown in FIG. 8B, the HMC with MPEGTMS treatment had no cells attached to the outside, whereas the cells on the inner surface show normal morphology of 3T3 fibroblasts. HMCs without MPEGTMS treatment has cells attached on both the inner and outer surfaces, as shown in FIG. 8C.

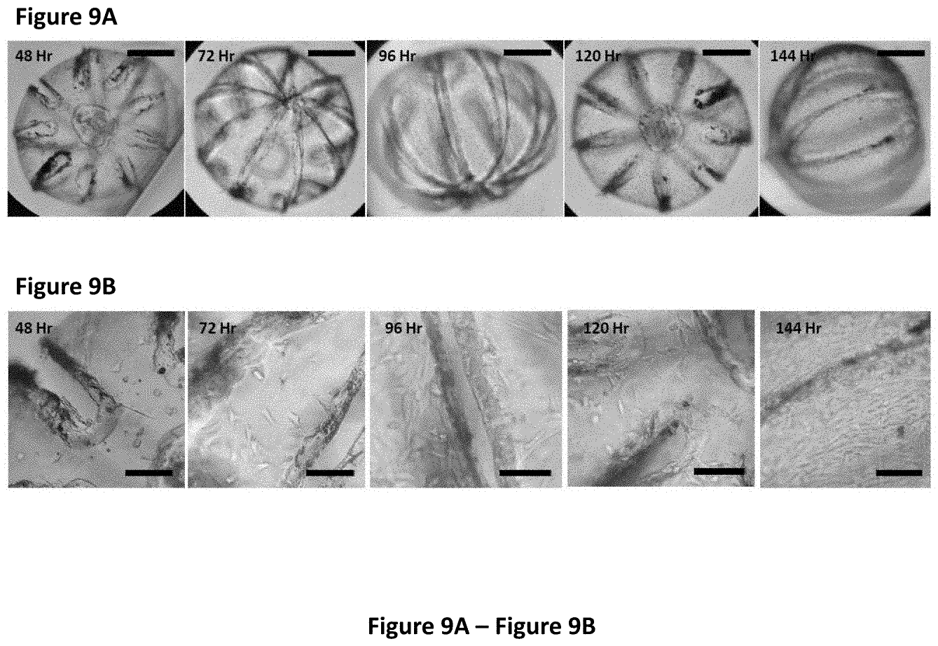

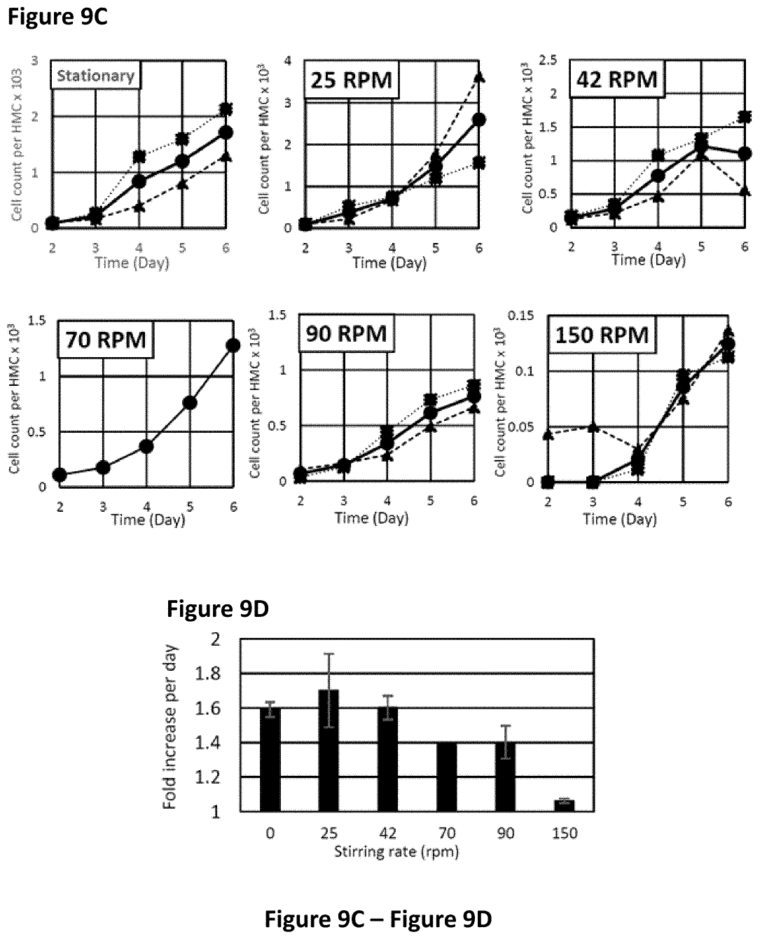

HMCs enabled the dynamic culture of NIH/3T3 fibroblasts as shown in 9A through FIG. 9D. The phase contrast images of the cells in the HMCs are shown in FIG. 9A and FIG. 9B. The fibroblasts inside the HMCs exhibited a multipolar and elongated shape, which is a typical morphology of actively proliferating fibroblasts. The proliferation rate of the fibroblasts in HMCs were characterized as shown in FIG. 9C. The solid lines show the average cell number in each stirring condition while the dashed lines show the results from individual experiments. Active proliferation of the cells were observed over 6 days of culture with continuous stirring up to 90 rpm (revolution per minutes). In all of the conditions, the fibroblasts showed exponential growth over their culturing period except for one data point on the final day at 42 rpm. The expansion rate of fibroblasts for each stirring speed was calculated, as shown in FIG. 9D. The expansion rate was stable up to 42 rpm and shows a slight decline at 70 and 90 rpm. It decreased significantly at 150 rpm due to the elevated shear stress caused by very vigorous stirring. The fold increase per day at 25 rpm was larger than the static conditions, due to increased diffusion of nutrients and gases by stirring. During the 6 days of culture, the total cell number increased by 26.7, 29.7, 28, 7.1, 7.3, and 1.5 times for the stationary culture, 25 rpm, 42 rpm, 70 rpm, 90 rpm, and 150 rpm, respectively.

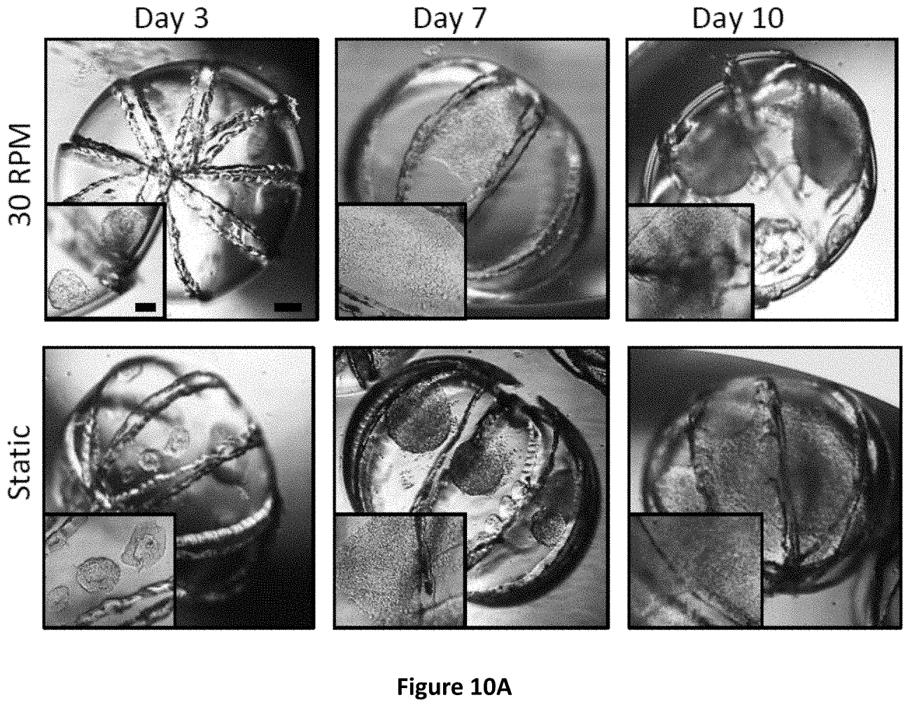

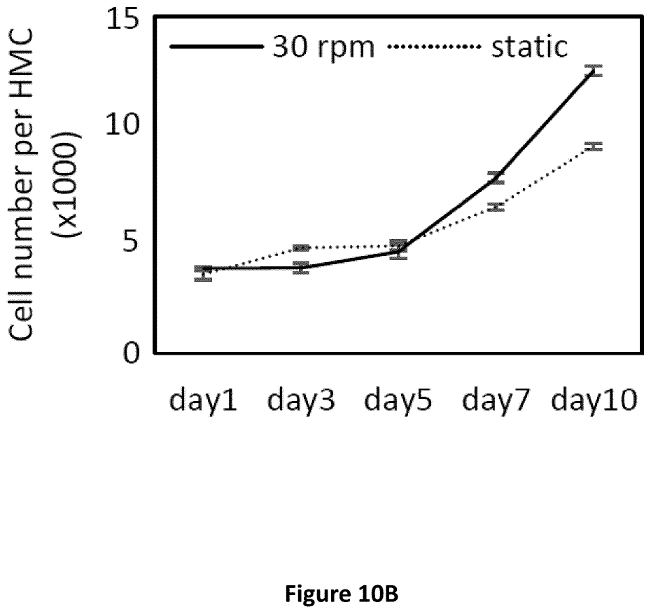

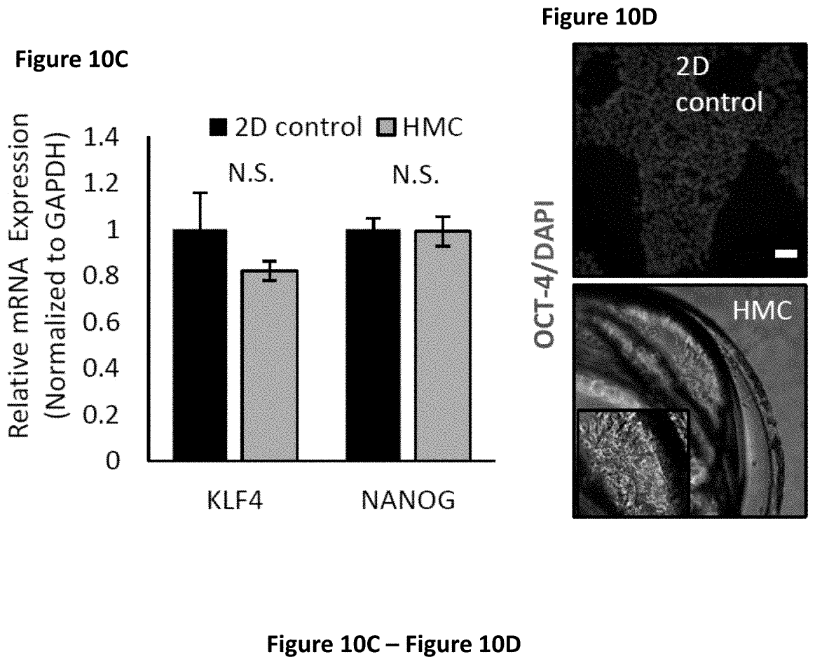

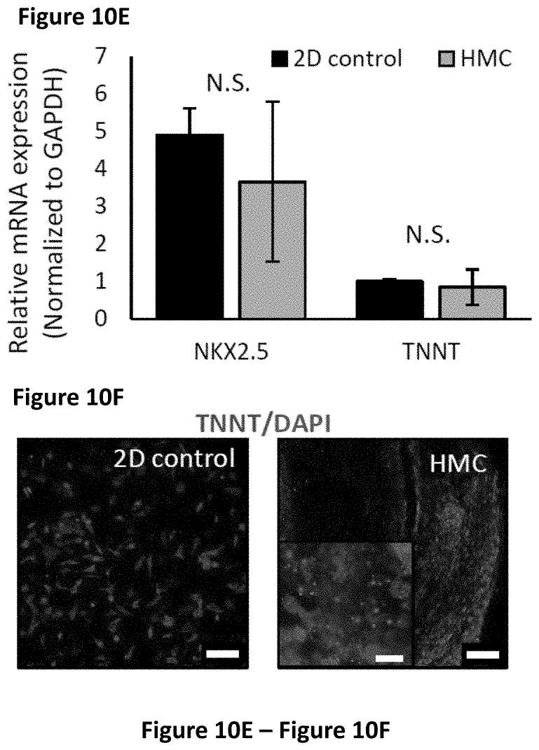

The recent advancements in stem cell research has introduced hiPSCs as a valuable cell source for acquiring various human-origin cell types without the ethical issues embryonic stem cells carry. hiPSCs are especially valuable as a source for cell types that are not available from primary sources, such as cardiomyocytes. The present study cultured and expanded hiPSCs in HMCs and differentiated them to hiPSC-derived CMs (iCMs) to explore the potential of HMCs for large-scale production of hiPSC-derived cells. The hiPSCs were seeded to Geltrex coated HMCs and achieved successful attachment (FIG. 10A). The selective coating of the inner surface of HMCs prevented cell attachment to the outside surface, as shown by the smooth outer surface over a period of 10 days as shown in FIG. 10A. Under both static and dynamic conditions, the hiPSCs were able to form the characteristic colony-like phenotype (FIG. 10A) and proliferate (FIG. 10B) inside the HMCs. For dynamic culture of hiPSCs, stirring at 30 rpm was used, considering the fold increase per day in NIH/3T3. Since the highest population increase was achieved on 25 and 42 rpm, it was determined that a speed within this range was to be used. As indicated by the cell number increase throughout the 10-day culture period, the hiPSCs can be expanded in HMCs under dynamic conditions. The hiPSCs require the formation of colonies for active proliferation. As such, the number of the hiPSC in the HMCs increased slowly in an earlier phase of the expansion, as shown in day 1 and day 3. Once the hiPSC formed colonies, they proliferated exponentially. The proliferation dynamics can be further optimized by varying the initial seeding densities. Importantly, when compared to static culture conditions, incubation under dynamic conditions induced a faster population growth, similarly to NIH/3T3. On day 7 of culture, the cell number per HMC was 6515±145 under the static condition and 7799±213 under the dynamic condition at 30 rpm. This difference was even higher when day 10 was reached; the cell number per HMC was calculated to be 9212±148 under the static condition, and measured to be 12568±276 under the dynamic condition at 30 rpm, suggesting that HMCs are suitable for hiPSC culture and that dynamic conditions provide enhanced cell proliferation through better diffusion of nutrients and gases. The healthy hiPSC phenotype was characterized by investigating the pluripotency of the hiPSCs. hiPSCs were determined to maintain their pluripotency in HMCs quantitatively and qualitatively by using q-RT-PCR and immunostaining, respectively, as shown in FIG. 10C and FIG. 10D. The hiPSCs cultured in HMCs showed similar mRNA expression of pluripotency markers KLF-4 and NANOG in comparison to hiPSCs cultured on conventional 2-D culture surfaces (FIG. 10C).

Similarly, the protein expression of another pluripotency marker, OCT-4, was comparable between hiPSCs cultured in HMCs and in 2-D conventional culture surfaces (FIG. 10D). Overall, these results demonstrate that HMCs are suitable platforms for fast and successful expansion of hiPSCs. One of the crucial needs of stem cell research, tissue engineering, and regenerative medicine is large-scale and cost-effective production of mature and functional iCMs. Therefore, after characterizing the hiPSC growth and functionality, hiPSCs were differentiated in HMCs using a previously established protocol. At day 12 of differentiation, beating was observed in the HMCs, demonstrating successful differentiation. In addition, the iCMs were characterized for cardiac specific marker expression on both mRNA and protein levels. The q-RT-PCR results showed that the iCMs expressed NKX2.5 and TNNT at comparable levels to iCMs cultured on conventional 2-D surfaces. Similarly, the positive immunostaining against TNNT in both HMCs and 2-D cultures indicated that differentiation in HMCs allows for production of functional iCMs.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A hollow microcarrier comprising a thin shell forming a three-dimensional structure having a hollow interior, the structure having a shape selected from the group consisting of: a sphere, an elongated sphere, a cylinder, a spheroid, and a polyhedron.

2. The hollow microcarrier of claim 1, wherein the shell comprises one or more holes, gaps, or apertures accessing the hollow interior.

3. The hollow microcarrier of claim 1, wherein the shell comprises a plurality of elongate leaflets, each leaflet having a proximal end and a distal end, wherein the plurality of leaflets are joined to each other at their proximal ends in a radial pattern, and wherein the distal ends of the plurality of leaflets curl towards each other to form a substantially spherical shape having a hollow interior.

4. The hollow microcarrier of claim 3, comprising between 3 and 10 leaflets.

5. The hollow microcarrier of claim 1, wherein the shell comprises a plurality of elongate leaflets, each leaflet having a proximal end and a distal end, wherein the plurality of leaflets are joined to each other at their proximal ends in a first and a second radial pattern, wherein the first and second radial patterns are joined to each other by the distal end of a leaflet, and wherein the distal ends of the leaflets curl towards each other such that the first radial pattern and the second radial pattern each form a hemisphere of a substantially spherical shape having a hollow interior.

6. The hollow microcarrier of claim 1, wherein the shell comprises a plurality of elongate leaflets, each leaflet defining a gore segment having opposing ends and a central region, wherein each leaflet is joined to an adjacent leaflet at the central region in a linear array, and wherein the opposing ends of the leaflets curve towards each other to form a substantially spherical shape having a hollow interior.

7. The hollow microcarrier of claim 1, wherein the shell comprises a rectangular leaflet joined to two circular leaflets, wherein the leaflets curve towards each other such that the rectangular leaflet forms a curved outer surface and the circular leaflets form opposing ends of a substantially cylindrical shape having a hollow interior.

8. The hollow microcarrier of claim 1, wherein the shell comprises a plurality of polygonal leaflets joined to each other, wherein the leaflets curve towards each other to form a substantially polyhedral shape having a hollow interior.

9. The hollow microcarrier of claim 1, constructed from a layer of a first material bonded to a layer of a second material.

10. The hollow microcarrier of claim 9, wherein the layer of the first material and the layer of the second material are under different amounts of tensile stress or different amounts of compressive stress.

11. The hollow microcarrier of claim 10, wherein the different amounts of tensile stress or different amounts of compressive stress are caused by the layer of the first material and the layer of the second material having different coefficients of thermal expansion.

12. The hollow microcarrier of claim 10, wherein the different amounts of tensile stress or different amounts of compressive stress are caused by the layer of the first material and the layer of the second material being fabricated at different processing temperatures.

13. The hollow microcarrier of claim 10, wherein the different amounts of tensile stress or different amounts of compressive stress are caused by the layer of the first material and the layer of the second material having different swelling ratios.

14. The hollow microcarrier of claim 1, having a diameter between about 50 μm and 10 mm.

15. The hollow microcarrier of claim 9, wherein the first material is a mix of Sylgard 184 and Sylgard 3-6636 in a 5:1:3:3 ratio of Base _{Sylgard 184}: Curing agent _{Sylgard 184}: Part-A _{Sylgard 3-6636}: Part-B _{Sylgard 3-6636}.

16. The hollow microcarrier of claim 9, wherein the second material is a mix of Sylgard 184 and Xiameter-200M in a 4:1:1 ratio of Base _{Sylgard 184}: Curing agent _{Sylgard 184}: Xiameter.

17. The hollow microcarrier of claim 1, wherein the shell comprises one or more markings selected from the group consisting of: letters, numbers, shapes, symbols, barcodes, Quick Response (QR) codes, images, and combinations thereof.

18. A method of fabricating hollow microcarriers, the method comprising the steps of:
depositing a layer of sacrificial material on a substrate;
depositing a layer of a first material on the layer of sacrificial material;
depositing a layer of a second material on the layer of the first material;
engraving one or more hollow microcarrier patterns into the layer of sacrificial material, the layer of the first material, and the layer of the second material;
applying one or more surface treatments to the layer of the second material; and
removing the layer of sacrificial material to release the layer of the first material and the layer of the second material from the substrate.

19. The method of claim 18, wherein the sacrificial material is AZ-9260 photoresist spin-coated on a substrate at 1300 rpm for 10 seconds to achieve a layer thickness of 13 μm and baked at 140° C. for 1 hour.

20. The method of claim 18, wherein the substrate is a flat piece of silicon.

21. The method of claim 18, wherein the first material is a mix of Sylgard 184 and Sylgard 3-6636 in a 5:1:3:3 ratio of Base _{Sylgard 184}: Curing agent _{Sylgard 184}: Part-A _{Sylgard 3-6636}: Part-B _{Sylgard 3-6636} that is spin-coated on the sacrificial material at 2000 rpm for 3 minutes to achieve a layer thickness of 18 μm and baked at 40° C. for 12 hours.

22. The method of claim 18, wherein the second material is a mix of Sylgard 184 and Xiameter-200M in a 4:1:1 ratio of Base _{Sylgard 184}: Curing agent _{Sylgard 184}: Xiameter that is spin-coated on the first material at 1300 rpm for 2 minutes to achieve a layer thickness of 19 μm and baked at 130° C. for 3 hours.

23. The method of claim 18, wherein the one or more surface treatments includes a corona discharge treatment that renders portions of the hollow microcarrier hydrophilic or hydrophobic.

24. The method of claim 18, wherein the one or more surface treatments includes a coating of a cell growth promoting composition.

25. The method of claim 18, further comprising a step of applying one or more markings on the sacrificial material, the first material, the second material, and combinations thereof using photolithography, stereolithography, or laser etching.

26. The method of claim 25, wherein the one or more markings are selected from the group consisting of: letters, numbers, shapes, symbols, barcodes, Quick Response (QR) codes, images, and combinations thereof.

27. A method of culturing cells using hollow microcarriers, the method comprising the steps of:
adding an amount of hollow microcarriers to a suspension of cells;
shifting the hollow microcarriers between a closed configuration, an open configuration, and back to a closed configuration in the suspension of cells to introduce cells into the hollow microcarriers;
incubating the hollow microcarriers under static conditions;
incubating the hollow microcarriers under dynamic conditions; and
shifting the hollow microcarriers from a closed configuration to an open configuration to harvest the cells from the hollow microcarriers.

28. The method of claim 27, wherein the hollow microcarriers are shifted between the open configuration and the closed configuration using thermal actuation or mechanical force.
